(19) United States
(12) Patent Application Publication
(10) Pub. No.: US 2025/0114420 A1
(43) Pub. Date: Apr. 10, 2025

(54) ANTIMICROBIAL NATURAL COMPOSITIONS

(71) Applicant: BIO-ACTIVES SYNERGIO LTD, Jerusalem (IL)

(72) Inventors: Tova SILBERSTEIN, Jerusalem (IL); Alexander BESONOV, Rehovot (IL); Rachel LUTZ, Gush etzion (IL); Sabrina BARCHICHAT, Jerusalem (IL); Tal SHALEV, Tzur Hadassah (IL); Tamar DVASH, Kibbutz Tzuba (IL); Arie AFEK, Kfar Saba (IL); Arye Shalom KAGAN, Jerusalem (IL); Eyal SIGAWI, Rehovot (IL)

(21) Appl. No.: 18/833,149

(22) PCT Filed: Jan. 27, 2022
(86) PCT No.: PCT/IL2022/050124

§ 371 Date: Jul. 25, 2024

(51) Int. Cl. A61K 36/48; A01N 65/08; A01P 1/00; A61K 8/9789; A61K 31/351; A61K 36/36; A61K 36/537; A61K 36/54; A61K 36/575; A61K 36/61; A61K 36/65; A61K 36/71; A61K 36/73; A61K 36/77; A61K 36/82; A61K 36/899; A61K 45/06; A61P 31/04; A61P 31/10; A61Q 17/00

(52) U.S. Cl. CPC A61K 36/48; A01N 65/08; A01P 1/00; A61K 8/9789; A61K 31/351; A61K 36/36; A61K 36/537; A61K 36/54; A61K 36/575; A61K 36/61; A61K 36/65; A61K 36/71; A61K 36/73; A61K 36/77; A61K 36/82; A61K 36/899; A61K 45/06; A61P 31/04; A61P 31/10; A61Q 17/005

(57) ABSTRACT

The present disclosure generally concerns antimicrobial natural compositions that are based on combinations of extracts, e.g. plant extracts. More specifically, the present disclosure provides antimicrobial compositions comprising at least one saponin extract, an extract from at least one plant species selected from the genus Styrax (a Styrax extract), the genus Myroxylon (a Miroxylon extract), the genus Myrocarpus (a Myrocarpus extract) and mixtures thereof, and at least one additional plant extract from an additional plant source, the saponin extract being from a saponin source different from said plant species of the genera Styrax, Myroxylon, or Myrocarpus, and from said additional plant source.

### TECHNOLOGICAL FIELD

The present disclosure generally concerns antimicrobial natural compositions that are based on combinations of extracts, e.g. plant extracts.

### BACKGROUND

Public concern about the safety of synthetic preservatives used in cosmetic and foods, especially regarding their accumulation and subsequent health effect, have driven health authorities to reduce the applied concentrations or even ban synthetic preservatives. Alternatives such as plant antimicrobial substances are in the focus of many researches, however due to low potency, narrow range and high prices, they are rarely used to replace synthetic preservatives.

### REFERENCES

References considered to be relevant as background to the presently disclosed subject matter are listed below:
[1]“The saponins—polar isoprenoids with important and diverse biological activities” A. Osbourn, et al., Nat. Prod. Rep., 2011, 28, 1261
[2] WO 2000/072861
[3] WO 1998/048768
[4] US 2006/0018867
[5] WO 2009/153800
[6] WO 2012/07719
[7] WO 2012/077120
[8] CN101085222
[9] WO 2001/47481
[10] WO 2017/191629

### GENERAL DESCRIPTION

Although saponin exhibits preservation properties, such may often prove to be insufficient against various microbiological contaminants. The inventors of the invention disclosed herein have surprisingly found that compositions of at least one saponin material and extracts of various plant species exhibit biological activity, e.g. antimicrobial activity, which is superior to the activity demonstrated for each component individually and which is at least comparable, and at times even superior, to chemical (i.e. non-natural) alternatives known for the same use.

Thus, in one of its aspects, the present disclosure provides antimicrobial composition comprising at least one saponin extract, an extract from at least one plant species selected from the genus Styrax (a Styrax extract), the genus Myroxylon (a Miroxylon extract), the genus Myrocarpus (a Myrocarpus extract) and mixtures thereof, and at least one additional plant extract from an additional plant source, the saponin extract being from a saponin source different from said plant species of the genera Styrax, Myroxylon, or Myrocarpus, and from said additional plant source.

In another of its aspects, the present disclosure provides antimicrobial composition comprising at least one saponin extract, at least one plant extract obtained from at least one plant species of the genus Styrax, and at least one additional plant extract from an additional plant source, the saponin extract being from a saponin source different from said plant species of the genus Styrax and said additional plant source.

In another aspect, the present disclosure provides a composition comprising saponin extract, at least one plant extract from at least one plant species selected from the genus Styrax, Myroxylon and/or Myrocarpus and mixtures thereof, and at least one additional plant extract from an additional plant source, the saponin extract being from a saponin source different from said plant species of the genus Styrax, Myroxylon and Myrocarpus and from said additional plant source, the Styrax, Myroxylon and/or Myrocarpus extract containing at least one of cinnamic acid, cinnamic acid derivatives and/or benzoic acid derivatives.

Another aspect of this disclosure provides antimicrobial composition comprising at antimicrobial effective amount of a mixture of agents, the mixture of agents consisting of at least one saponin extract, at least one plant extract from at least one plant species selected from the genus Styrax, Myroxylon and/or Myrocarpus and mixtures thereof, and at least one additional plant extract from an additional plant source, the saponin extract being from a saponin source different from said at least one plant species and from said additional plant source.

Yet a further aspect provides for an add-on composition consisting of a combination of at least one saponin extract, at least one plant extract from at least one plant species selected from the genus Styrax, Myroxylon and/or Myrocarpus and mixtures thereof, and at least one additional plant extract from an additional plant source, the saponin extract being from a saponin source different from said at least one plant species and from said additional plant source.

In another of its aspects, the disclosure provides a cosmetic or cleansing formulation comprising compositions of the present disclosure as defined in the various embodiments herein.

In yet another aspect, the disclosure provides a preservative formulation comprising the compositions of the disclosure as described herein.

As used herein, the term extract refers to an active ingredient or fraction isolated from a plant, typically by solvent extraction, although other extraction techniques known per-se are also contemplated. The extraction procedure for obtaining any of the plant extracts employed in accordance with this disclosure, unless otherwise indicated, may be carried out in any commonly used technique and variation known in the art.

Extracts of the plant species used for preparing the compositions of this disclosure may be prepared prior to formulation, in advance of formulation or may be commercially available. The extracts may be used without further purification.

Saponin material, as used herein is at least one naturally obtained saponin compound, as known in the art. When isolated from a natural source, the saponin material may be used in its substantially pure form (namely at least 85%, 87%, 92%, 95%, or 98% purity), or may be used as a saponin-containing extract (also referred to herein for the purpose of brevity as “saponin extract”) isolated by a method known in the art.

In accordance with the present disclosure, the saponin-containing extract contains at least between 0.2% and 95 wt % saponins, out of the total weight of the dry content of the extract. In some embodiments, the extract used in accordance with the present disclosure comprises between 0.2% and 99 wt % saponins out of the total weight of the dry content of the extract.

In some embodiments, the saponin extract used in the compositions may comprise between about 10% and about 80 wt % saponins out of the total weight of the dry content of the extract. In other embodiments, the saponin extract may comprise between about 10% and about 60 wt % saponins, between about 10% and about 50 wt % saponins, between about 10% and about 40 wt % saponins, between about 10% and about 30 wt % saponins, or even between about 10% and about 20 wt % saponins out of the total weight of the dry content of the extract. In some embodiments, the saponin extract comprises between about 0.2% and about 10 wt % saponins out of the total weight of the dry content of the extract.

When isolated from a natural source, the saponin extract may be used in its substantially pure form (namely at least 85%, 87%, 92%, 95%, or 98% purity).

The saponin-containing extract may be obtained from any natural source known to comprise saponins. Such natural source may be a plant source, some of which are detailed infra, and also from non-plant sources such as animal sources and marine organisms, such as starfish and sea cucumbers. In some embodiments of this disclosure, the saponins are extracted from a plant source, naturally grown or genetically modified to have high saponin content. In some embodiments, the saponin material is obtained by extraction from a plant source by employing a solvent, water, alcohol or a water/alcohol solution. In some embodiments, the alcohol is ethanol or methanol.

The saponin material may be obtained from a plant source. The plant source may be selected from shikakai, soyabeans, beans, peas (Pisum sativum), lucerne, tea, spinach, sugar beet, quinoa, liquorice, sunflower, horse chestnut, ginseng, oats, capsicum peppers, aubergine, tomato seed, alliums, asparagus, yam, fenugreek, yucca and ginseng, lucerne, mung beans, Bupleurum falcatum, Camellia oleifera, Camellia sinensis, Desmodium adscendens, Gypsophila, Panax quinqufolius, Panax japonicas, Quillaja saponaria, Sapindus delavayi, Sapindus mukorossi, Sapindus marginatus, Sapindus saponaria, Sapindus trifoliatus, Saponaria officinalis, and Yucca schidigera or any mixture thereof.

In some embodiments, the saponin extract is obtained from a plant source selected from Camellia oleifera, Camellia sinensis, Quillaja saponaria, Sapindus mukorossi, Sapindus saponaria, and Saponaria officinalis or any mixture thereof. In other embodiments, the saponin extract is obtained from Camellia oleifera, Quillaja saponaria and/or Sapindus mukorossi. Saponin containing material may be purified by any means known in the art, including filtration, centrifugation, re-crystallization, distillation, adsorption, chromatographic methods, fractionation, etc.

The saponin extract may be obtained from any part of the plant, including leaves, stems, roots, bulbs, blossom and fruit (including the skin, flesh and seed of the fruit). In some embodiments, the extracts are obtained from the pericarp of Sapindus mukorossi, or the seed meal of Camellia oleifera.

Compositions of the disclosure may comprise, by some embodiments, at least 0.001 wt % saponin. In some embodiments, the compositions comprise between about 0.01 and 2 wt %, between about 0.01 and 1.5 wt %, or even between about 0.01 and 1 wt % saponin.

As noted above, in addition to the saponin extract, the composition comprises an extract from at least one plant species selected from the genus Styrax (a Styrax extract), the genus Myroxylon (a Miroxylon extract), the genus Myrocarpus (a Myrocarpus extract) and mixtures thereof, and at least one additional plant extract from an additional plant source. It is of note that when the saponin is obtained from a plant extract, the saponin extract is from a saponin source different from said plant species of the genus Styrax and said additional plant source.

The genus Styrax contains a group of small trees or shrubs in the family Styracaceae, which often secrete a gum-like resin containing at least benzoic acid, coniferyl benzoate and other compounds. The gum-like resin or tree bark is often called “benzoin resin”. It should be appreciated that the extract may be an extract of more than one plant selected within the genus.

In some embodiments, the plant of the genus Styrax may be selected from Styrax agrestis, Styrax americanus, Styrax argenteus, Styrax argentifolius, Styrax argyrophyllus, Styrax bashanensis, Styrax benzoides, Styrax benzoin, Styrax calvescens, Styrax camporum, Styrax chinensis, Styrax chrysocarpus, Styrax confuses, Styrax crotonoides, Styrax dasyanthus, Styrax faberi, Styrax ferax, Styrax ferrugineus, Styrax formosanus, Styrax foveolaria, Styrax fraserensis, Styrax grandiflorus, Styrax grandifolius, Styrax hainanensis, Styrax hemsleyanus, Styrax hookeri, Styrax huanus, Styrax jaliscana, Styrax japonicas, Styrax limpritchii, Styrax litseoides, Styrax loxensis, Styrax macranthus, Styrax macrocarpus, Styrax martii, Styrax mathewsii, Styrax obassia, Styrax odoratissimus, Styrax officinalis, Styrax paralleloneurus (Sumatra benzoin), Styrax parvifolium, Styrax perkinsiae, Styrax peruvianum, Styrax philadelphoides, Styrax platanifolius, Styrax pohlii, Styrax portoricensis, Styrax redivivus, Styrax roseus, Styrax rugosus, Styrax schweliense, Styrax serrulatus, Styrax shiraianum, Styrax socialis, Styrax suberifolius, Styrax supaii, Styrax tafelbergensis, Styrax tolu, Styrax tonkinensis (Siam Benzoin), Styrax veitchiorum, Styrax vilcabambae, Styrax wilsonii, Styrax wuyuanensis, Styrax zhejiangensis and mixtures thereof.

In some embodiments, the plant of the genus Styrax is selected from Styrax paralleloneurus (Sumatra benzoin), Styrax tonkinensis (Siam Benzoin), Styrax formosanus, Styrax peruvianum, Styrax tolu and mixtures thereof.

In some other embodiments, the plant of the genus Styrax is Styrax paralleloneurus (Sumatra benzoin).

In other embodiments, the plant of the genus Styrax is Styrax tonkinensis (Siam Benzoin).

In further embodiments, the plant of the genus Styrax is Styrax formosanus.

In some further embodiments, the plant of the genus Styrax is Styrax peruvianum.

In yet further embodiments, the plant of the genus Styrax is selected Styrax tolu.

According to some embodiments, the Styrax extract is obtained by mixing the resin and/or bark with a suitable solvent, typically a hydrophobic solvent, such as at least one oil (e.g. paraffinic oil, triglycerides, non-triglyceride oils, etc.), pentane, hexane, cyclohexane, heptane, octane, dichloromethane, dichloroethane, chloroform, etc.

In other embodiments, each of the plant extracts (i.e. either or both of the saponin and Styrax extracts) is obtained commercially.

The genus Myroxylon contains a group of small trees in the family Fabaceae, which often secrete a gum-like resin containing at least benzoic acid, coniferyl benzoate and other compounds.

In some embodiments, the at least one plant extract is a Myroxylon extract, obtained from the plant of the genus Myroxylon selected from Myroxylon balsamum and Myroxylon peruiferum.

The Myroxylon extract can be obtained from the resin or bark of the plant.

The genus Myrocarpus contains a group of small trees also in the family Fabaceae, and its gum-like resin contains at least benzoic acid, coniferyl benzoate and other compounds.

In some embodiments, the at least one plant extract is a Myrocarpus extract, obtained from the plant of the genus Myrocarpus selected from Myrocarpus fastigiatus, Myrocarpus frondosus, Myrocarpus leprosus and Myrocarpus venezuelensis. According to some embodiments, the Myrocarus extract is a Myrocarpus fastigiatus extract. The Myrocarpus extract can be obtained from the resin or bark of the plant.

Compositions of this disclosure may comprise, by some embodiments, at least 0.001 wt % plant extract from the genus Styrax, Myroxylon and/or Myrocarpus. In some embodiments, the compositions comprise between about 0.001 and 5 wt % of the plant extract. In some embodiments, the compositions comprise between about 0.001 and 4 wt %, between about 0.001 and 3 wt %, or even between about 0.001 and 2 wt % of the plant extract. In other embodiments, the compositions comprise between about 0.005 and 5 wt %, between about 0.01 and 5 wt %, between about 0.03 and 5 wt %, or even between about 0.05 and 5 wt % of the plant extract. In some other embodiments, the compositions comprise between about 0.005 and 4 wt %, between about 0.01 and 3 wt %, between about 0.03 and 2.5 wt %, or even between about 0.05 and 2 wt % of plant extract.

The inventors have found that natural extracts of Styrax (as well as Myroxylon and Myrocarpus) contain various compounds which provide, once formulated with the saponin-containing extract, comparable or even superior preservation properties compared to chemical (i.e. non-natural) alternatives are obtained.

Thus, the present disclosure provides a composition comprising saponin extract, an extract from at least one plant species selected from the genus Styrax, Myroxylon and/or Myrocarpus and mixtures thereof, and at least one additional plant extract from an additional plant source, the saponin extract being from a saponin source different from said plant species of the genus Styrax, Myroxylon and Myrocarpus and from said additional plant source, the Styrax, Myroxylon and/or Myrocarpus extract containing at least one of cinnamic acid, cinnamic acid derivatives and/or benzoic acid derivatives.

In some embodiments, the total amount of cinnamic acid, cinnamic acid derivatives and benzoic acid derivatives in the at least one plant extract is at least 0.001, 0.01, or even at least 0.1 wt %. In other embodiments, the total amount of cinnamic acid, cinnamic acid derivatives and benzoic acid derivatives in the at least one plant extract may be at most 10 wt %. In some other embodiments, the total amount of cinnamic acid, cinnamic acid derivatives and benzoic acid derivatives in the at least one plant extract may be between about 0.001 and 10 wt %, between about 0.01 and 10 wt %, between about 0.1 and 10 wt %, or even between about 1 and 10 wt %.

The term derivative refers to a chemically modified compound derived from a parent compound (e.g. cinnamic acid or benzoic acid) that differs from the parent compound by one or more elements, substituents and/or functional groups such that the derivative has the same or similar properties/activities as the parent compound, as defined herein.

In some embodiments, the cinnamic acid derivative is selected from P-coumaryl cinnamate, coniferyl cinnamate, cinnamyl cinnamate, benzyl cinnamate, cinnamic acid esters, etc., and combinations thereof.

In other embodiments, the benzoic acid derivative is selected from coniferyl benzoate, cinnamyl benzoate, P-coumaryl benzoate, benzoic acid esters, etc., and combinations thereof.

The Styrax extract may further comprise various terpenes and terpenoids, as well as other phenolic derivatives, such as pinoresinol.

It is of further note that the derivatives may be obtained from a natural source, or may be artificially synthesized. Thus, in another one of its aspects, the present disclosure provides a composition comprising saponin extract and a formulation comprising at least one of cinnamic acid, cinnamic acid derivatives and/or benzoic acid derivatives.

In some embodiments, cinnamic acid, cinnamic acid derivatives and/or benzoic acid derivatives may be components of a Styrax extract from the resin or bark of at least one plant species of the genus Styrax (such that the saponin extract of the composition is from a saponin source different from said plant species of the genus Styrax), or a components of a Myroxylon extract from the resin or bark of at least one plant species of the genus Myroxylon, and/or a components of a Myrocarpus extract from the resin or bark of at least one plant species of the genus Myrocarpus.

Generally, in the compositions of this disclosure, the weight-to-weight ratio (wt/wt) between the saponin extract and the at least one plant extract may range between 1:50 and 50:1 (saponin material:plant extract). In some embodiments, the weight-to-weight ratio between the saponin extract and the at least one plant extract is about 1:50, 1:45, 1:30, 1:25, 1:20, 1:15, 1:10, 1:8, 1:7, 1:6, 1:5, 1:4, 1:3, 1:2, 1:1, 2:1, 3:1, 4:1, 5:1, 6:1, 7:1; 8:1, 10:1, 15:1, 20:1, 25:1, 30:1, 35:1, 40:1, 45:1, or about 50:1.

In other embodiments, the weight ratio between the saponin extract and the at least one plant extract may be between 1:20 and 20:1. In other embodiments, the weight ratio between the saponin extract and the at least one plant extract may be between 1:10 and 10:1. In some other embodiments, the weight ratio is between 1:5 and 5:1.

By some embodiments, the weight-to-weight ratio (wt/wt) between the additional plant extract and the at least one plant (i.e. Styrax, Myroxylon and/or Myrocarpus) extract may range between about 1:400 and 400:1, between about 200:1 and 1:200, or even between about 1:100 and 100:1. In some embodiments, the weight-to-weight ratio between the additional plant extract and the at least one plant extract is about 1:100, 1:90, 1:80, 1:70, 1:60, 1:50, 1:40, 1:30, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1; 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1 or about 100:1.

In other embodiments, the weight ratio between the additional plant extract and the at least one plant extract may be between 1:25 and 25:1. In some other embodiments, the weight ratio is between 1:20 and 20:1.

The content of the additional plant extract in the composition may be, by some embodiments, at least 0.001 wt %. In some embodiments, the content of the additional plant extract in compositions may be between about 0.005 and 2 wt %, between about 0.01 and 1.5 wt % or even between about 0.02 and 1 wt %.

In the compositions of this disclosure, the weight-to-weight ratio (wt/wt) between the saponin extract and the additional plant extract may range between 1:200 and 200:1 (saponin material:additional plant extract). In some embodiments, the weight-to-weight ratio between the saponin extract and the additional plant extract is about 1:200, 1:175, 1:150, 1:125, 1:100, 1:75, 1:50, 1:45, 1:30, 1:25, 1:20, 1:15, 1:10, 1:8, 1:7, 1:6, 1:5, 1:4, 1:3, 1:2, 1:1, 2:1, 3:1, 4:1, 5:1, 6:1, 7:1; 8:1, 10:1, 15:1, 20:1, 25:1, 30:1, 35:1, 40:1, 45:1, 50:1, 75:1, 100:1, 125:1, 150:1, 175:1, or even between 200:1.

In other embodiments, the weight ratio between the saponin extract and the additional plant extract may be between 100:1 and 1:100. In some other embodiments, the weight ratio is between 50:1 and 1:50. In yet other embodiments, the weight ratio is between 20:1 and 1:20. In some further embodiments, the weight ratio is between 10:1 and 1:10.

According to some embodiments, the additional plant extract may be selected from extracts of at least one plant species of the genera Cinnamomum, Paeonia, Salvia, Magnolia, Commiphora, Boswellia, Dipterocarpus, Copaiba, Malpighia, Thymus, Origanum, Cymbopogon, Anethum, Syzygium, Nigella, Pimenta, as well as maltol extracted from a plant source, and mixtures thereof.

By some embodiments, the additional plant extract is selected from extracts of at least one plant species of the genera Cinnamomum, Paeonia, Magnolia, Pimenta, and Cymbopogon, and mixtures thereof.

By some other embodiments, the additional plant extract may be selected from Salvia, Commiphora, Boswellia, Dipterocarpus, Copaiba, Malpighia, Thymus, Origanum, Anethum, and Syzygium, as well as maltol extracted from a plant source, and mixtures thereof.

It should be understood that the additional extract may be an extract of more than one plant selected from the genera Cinnamomum, Paeonia, Salvia, Magnolia, Commiphora, Boswellia, Dipterocarpus, Copaiba, Malpighia, Thymus, Origanum, Cymbopogon, Anethum, Syzygium, Nigella, Pimenta, or from a different genus. It should be further that the present disclosure further contemplates compositions comprising mixtures of such extracts, whether prepared and formulated individually or prepared in one-pot from a mixture of plant sources (plant parts).

The genus Cinnamomum is a genus of evergreen aromatic trees and shrubs belonging to the laurel family, which includes, inter alia, the species Cinnamomum acuminatifolium, Cinnamomum acuminatissimum, Cinnamomum acutatum, Cinnamomum africanum, Cinnamomum aggregatum, Cinnamomum alainii, Cinnamomum alatum, Cinnamomum albiflorum, Cinnamomum aicinii, alexei, Cinnamomum aliberiii, Cinnamomum alternifolium, Cinnamomum altissimum, Cinnamomum ammnannii, Cinnamomum amoenum, Cinnamomum amplexicaule, Cinnamomum amplifolium, Cinnamomum anacardium, Cinnamomum andersonii, Cinnamonumn angustifolium, Cinnamomum angustitepalum, Cinnamomum antillarum, Cinnamomum appehianum, Cinnamomum arbusculum, Cinnamomum archboldianum, Cinnamomum areolatocostae, Cinnamomum areolatum, Cinnamomum arfakense, Cinnamomum argenteun, Cinnamomum arsenei, Cinnamomum asa-grayi, Cinnamomum assarnictum, Cinnamomum aubletii, Cinnamomum aureo-fulvum, Cinnamomum austral, Cinnamomum austro-sinense, Cinnamomum austro-yunnanense, Cinnamomum bahianum, Cinnamomum bahiense, Cinnamomum baileyanun, Cinnamomum baillonii, Cinnamomum balansae, Cinnamomum bamoense, Cinnamomum barbato-axillatum, Cinnamomum barbeyanum, Cinnamomum barlowii, Cinnamomum bartheifolium, Cinnamomum barthii, Cinnamomum bazania, Cinnamomum beccarii, Cinnamomum bejolghota, Cinnamomum bengalense, Cinnamomum biafranum, Cinnamomum bintulense, Cinnamomum bimanicum, Cinnamomum blumei, Cinnamomum bodinieri, Cinnamomum bonii, Cinnamomum bonplandii, Cinnamomum borneense, Cinnamomum bourgeauvianum, Cinnamomum boutonii, Cinnamomum brachythyrsum, Cinnamomum bractefoliaceum, Cinnamomum burmnannii, Cinnamomum canbodianum, Cinnamomum camphora, Cinnamomum cassia, Cinnamomum caudiferum, Cinnamomum cebuense, Cinnamomum chartophyllum, Cinnamomum citriodorum, Cinnamomum contractum, Cinnamomum culilawan, Cinnamomum dubium, Cinnamomum elegans, Cinnamomum filipes, Cinnamomum glanduliferum, Cinnamomum glaucescens, Cinnamomum ilicioides, Cinnamomum impressinervium, Cinnamomum iners, Cinnamomum japonicum, Cinnamomum javanicum, Cinnamomum jensenianum, Cinnamomum kanehirae, Cinnamomum kotoense, Cinnamomum kwangtungense, Cinnamomum liangii, Cinnamomum longepaniculatum, Cinnamomum longipetiolatum, Cinnamomum loureiroi, Cinnamomum mairei, Cinnamomum malabatrum, Cinnamomum mercadoi, Cinnamomum micranthum, Cinnamomum migao, Cinnamomum mollifolium, Cinnamomum oliveri, Cinnamomum osmophloeum, Cinnamomum ovalifolium, Cinnamomum parthenoxylon, Cinnamomum pauciflorum, Cinnamomum pedunculatum, Cinnamomum philippinense, Cinnamomum pingbienense, Cinnamomum pittosporoides, Cinnamomum platyphyllum, Cinnamomum porphyrium, Cinnamomum porrectum, Cinnamomun reticulatum, Cinnamomum rigidissimum, Cinnamomum saxatile, Cinnamomum sepjentrionale, Cinnamomum sinharajaense, Cinnamomum sintoc, Cinnamomum subavenium, Cinnamomum tamala, Cinnamomum tenuipilum, Cinnamomum tonkinense, Cinnamomum triplinerve, Cinnamomum tsangii, Cinnamomum validinerve, Cinnamomum verum, Cinnamomum virens, Cinnamomum walaiwarense, and Cinnamomum wilsonii.

In some embodiments, the Cinnamomum extract is an extract of Cinnamomum cassia. In other embodiments, the Cinnamomum extract is an extract of Cinnamomum cassia bark or twig.

The genus Paeonia is a group of flowering plants in the family Paeoniaceae, which includes, inter alia, the species Paeonia algeriensis, Paeonia anomala, Paeonia arietina, Paeonia broteri, Paeonia brownii, Paeonia californica, Paeonia canbessedesii, Paeonia clusii, Paeonia coriacea, Paeonia corsica, Paeonia daurica, Paeonia emodi, Paeonia intermedia, Paeonia kesrouanensis, Paeonia lactiflora, Paeonia nairei, Paeonia mascula, Paeonia obovate, Paeonia officinalis, Paeonia parnassica, Paeonia peregrina, Paeonia sverniana, Paeonia tenuifolia, Paeonia veitchii, Paeonia decomposita, Paeonia delavayi, Paeonia jishanensis, Paeonia ludlowii, Paeonia ostia, Paeonia qiui, and Paeonia rockii.

In some embodiments, the Paeonia extract is an extract of Paeonia lactiflora. In other embodiments, the Paeonia extract is an extract of Paeonia lactiflora root.

The genus Salvia is a group of flowering plants in the family Lamiaceae, which includes, inter alia, the species Salvia absconditiflora, Salvia acuminata, Salvia adenocaulon, Salvia Adenophora, Salvia adenophylla, Salvia adiantifolia, Salvia adoxoides, Salvia aegyptiaca, Salvia aequidens, Salvia aequidistans, Salvia aerea, Salvia aethiopis, Salvia Africana, Salvia africana-lutea, Salvia alamosana, Salvia alariformis, Salvia alata, Salvia alatipetiolata, Salvia alba, Salvia albicalyx, Salvia albicaulis, Salvia albiflora, Salvia albimaculata, Salvia albocaerulea, Salvia alborosea, Salvia alexeenkoi, Salvia algeriensis, Salvia aliciae, Salvia altissima, Salvia alvajaca, Salvia amethystine, Salvia amissa, Salvia amplexicaulis, Salvia amplicalyx, Salvia amplifrons, Salvia anastomosans, Salvia anatolica, Salvia andreji, Salvia anguicoma, Salvia angulate, Salvia angustiarum, Salvia apiana, Salvia apparicii, Salvia appendiculate, Salvia arabica, Salvia aramiensis, Salvia arborescens, Salvia arbuscula, Salvia arduinervis, Salvia arenaria, Salvia areolate, Salvia argentea, Salvia Ariana, Salvia aridicola, Salvia aristate, Salvia arizonica, Salvia arthrocoma, Salvia articulata, Salvia aspera, Salvia asperata, Salvia asperifolia, Salvia assurgens, Salvia atrocalyx, Salvia atrocyanea, Salvia atropaenulata, Salvia atropatana, Salvia atropurpurea, Salvia atrorubra, Salvia aucheri, Salvia aurita, Salvia austriaca, Salvia austromelissodora, Salvia axillaris, Salvia axilliflora, Salvia ayavacensis, Salvia ayayacensis, Salvia aytachii, Salvia azurea, Salvia bahorucona, Salvia baimaensis, Salvia balansae, Salvia balaustina, Salvia baldshuanica, Salvia ballotiflora, Salvia ballsiana, Salvia bariensis, Salvia barrelieri, Salvia bazmanica, Salvia beckeri, Salvia benthamiana, Salvia betulifolia, Salvia bifidocalyx, Salvia biserrate, Salvia blancoana, Salvia blepharophylla, Salvia boegei, Salvia bogotensis, Salvia booleana, Salvia borjensis, Salvia bowleyana, Salvia brachyantha, Salvia brachyloba, Salvia brachyloma, Salvia brachyodon, Salvia brachyodonta, Salvia brachyphylla, Salvia bracteate, Salvia brandegeei, Salvia breviconnectivata, Salvia breviflora, Salvia brevilabra, Salvia brevipes, Salvia broussonetii, Salvia buchananii, Salvia bucharica, Salvia buchii, Salvia bulleyana, Salvia bullulata, Salvia caaguazuensis, Salvia cabonii, Salvia cabulica, Salvia cacaliifolia, Salvia cadmica, Salvia caespitosa, Salvia calaminthifolia, Salvia calcicole, Salvia californica, Salvia calolophos, Salvia camarifolia, Salvia campanulate, Salvia campicola, Salvia camporum, Salvia campylodonta, Salvia canariensis, Salvia candelabrum, Salvia candicans, Salvia candidissima, Salvia canescens, Salvia capillosa, Salvia carbonoi, Salvia cardenasii, Salvia cardiophylla, Salvia carduacea, Salvia carnea, Salvia cassia, Salvia castanea, Salvia cataractarum, Salvia caudata, Salvia cavaleriei, Salvia caymanensis, Salvia cedronella, Salvia cedrosensis, Salvia ceratophylla, Salvia cerradicola, Salvia chalarothyrsa, Salvia chamaedryoides, Salvia chamelaeagnea, Salvia chanryoenica, Salvia chapadensis, Salvia chapalensis, Salvia chiapensis, Salvia chicamochae, Salvia chienii, Salvia chinensis, Salvia chionantha, Salvia chionopeplica, Salvia chionophylla, Salvia chloroleuca, Salvia chorassanica, Salvia chrysophylla, Salvia chudaei, Salvia chunganensis, Salvia cilicica, Salvia cinica, Salvia cinnabarina, Salvia circinnata, Salvia clarendonensis, Salvia clausa, Salvia clevelandii, Salvia clinopodioides, Salvia coahuilensis, Salvia coccinea, Salvia cocuyana, Salvia codazziana, Salvia coerulea, Salvia cognata, Salvia colonica, Salvia columbariae, Salvia comayaguana, Salvia compar, Salvia compressa, Salvia compsostachys, Salvia concolor, Salvia confertiflora, Salvia congestiflora, Salvia connivens, Salvia consimilis, Salvia consobrina, Salvia corazonica, Salvia cordata, Salvia coriana, Salvia corrugate, Salvia costaricensis, Salvia costata, Salvia coulteri, Salvia crinigera, Salvia crucis, Salvia cruikshanksii, Salvia cryptoclada, Salvia cryptodonta, Salvia cuatrecasana, Salvia cubensis, Salvia curta, Salvia curticalyx, Salvia curtiflora, Salvia curviflora, Salvia cuspidate, Salvia cyanantha, Salvia cyanescens, Salvia cyanicalyx, Salvia cyanocephala, Salvia cyanotropha, Salvia cyclostegia, Salvia cylindriflora, Salvia cynica, Salvia dabieshanensis, Salvia darcyi, Salvia dasyantha, Salvia decumbens, Salvia decurrens, Salvia densiflora, Salvia dentata, Salvia deserta, Salvia deserti, Salvia desoleana, Salvia diamantina, Salvia dichlamys, Salvia dichroantha, Salvia digitaloides, Salvia discolor, Salvia disermas, Salvia disjuncta, Salvia divaricate, Salvia divinorum, Salvia dolichantha, Salvia dolomitica, Salvia dombeyi, Salvia dominica, Salvia dorisiana, Salvia dorrii, Salvia drobovii, Salvia drusica, Salvia drymocharis, Salvia dryophila, Salvia dugesiana, Salvia dumetorum, Salvia durantiflora, Salvia durifolia, Salvia duripes, Salvia ecbatanensis, Salvia ecuadorensis, Salvia eichleriana, Salvia eigii, Salvia eizi, Salvia ekimiana, Salvia elegans, Salvia elenevskyi, Salvia emaciate, Salvia engelmannii, Salvia eplingiana, Salvia eremophila, Salvia eremostachya, Salvia eriocalyx, Salvia eriophora, Salvia ernesti, Salvia erythropoda, Salvia erythrostephana, Salvia erythrostoma, Salvia espirito, Salvia euphratica, Salvia evansiana, Salvia exilis, Salvia expansa, Salvia exserta, Salvia fairuziana, Salvia falcata, Salvia farinacea, Salvia filicifolia, Salvia filifolia, Salvia filipes, Salvia firma, Salvia flaccida, Salvia flaccidifolia, Salvia flava, Salvia flocculosa, Salvia florida, Salvia fluviatilis, Salvia fominii, Salvia formosa, Salvia forreri, Salvia forsskaolei, Salvia foveolate, Salvia fracta, Salvia fragarioides, Salvia freyniana, Salvia frigida, Salvia fruticetorum, Salvia fruticosa, Salvia fruticulosa, Salvia fugax, Salvia fulgens, Salvia funckii, Salvia funerea, Salvia fusca, Salvia fuscomanicata, Salvia garedjii, Salvia gariepensis, Salvia garipensis, Salvia gattefossei, Salvia gesneriiflora, Salvia ghiesbreghtii, Salvia glabra, Salvia glabrata, Salvia glabrescens, Salvia glabricaulis, Salvia glandulifera, Salvia glechomifolia, Salvia glumacea, Salvia glutinosa, Salvia goldmanii, Salvia golneviana, Salvia gontscharowii, Salvia gonzalezii, Salvia gracilipes, Salvia graciliramulosa, Salvia grandifolia, Salvia grandis, Salvia granitica, Salvia gravida, Salvia greatae, Salvia greggii, Salvia grewiifolia, Salvia grisea, Salvia griseifolia, Salvia grossheimii, Salvia guadalujarensis, Salvia guaranitica, Salvia guarinae, Salvia haenkei, Salvia haitiensis, Salvia hajastana, Salvia halaensis, Salvia halophila, Salvia hamulus, Salvia handelii, Salvia hapalophylla, Salvia harleyana, Salvia hasankeyfensis, Salvia hatschbachii, Salvia haussknechtii, Salvia hayatae, Salvia hedgeana, Salvia heerii, Salvia heldreichiana, Salvia helianthemifolia, Salvia henryi, Salvia herbacea, Salvia herbanica, Salvia hermesiana, Salvia herrerae, Salvia heterochroa, Salvia heterofolia, Salvia heterotricha, Salvia hians, Salvia hidalgensis, Salvia hierosolymitana, Salvia hilarii, Salvia hillcoatiae, Salvia himmelbaurii, Salvia hintonii, Salvia hirsute, Salvia hirta, Salvia hirtella, Salvia hispanica, Salvia holwayi, Salvia honania, Salvia hotteana, Salvia huberi, Salvia humboldtiana, Salvia hupehensis, Salvia hydrangea, Salvia hylocharis, Salvia hypargeia, Salvia hypochionaea, Salvia hypoleuca, Salvia incumbens, Salvia incurvata, Salvia indica, Salvia indigocephala, Salvia infuscata, Salvia innoxia, Salvia inornate, Salvia insignis, Salvia insularum, Salvia integrifoli, Salvia interrupta, Salvia intonsa, Salvia involucrate, Salvia iodantha, Salvia iodophylla, Salvia ionocalyx, Salvia isensis, Salvia itaguassuensis, Salvia itatiaiensis, Salvia iuliana, Salvia jacobi, Salvia jaimehintoniana, Salvia jamaicensis, Salvia jaminiana, Salvia jamzadii, Salvia japonica, Salvia jaramilloi, Salvia jorgehintoniana, Salvia Judaica, Salvia jurisicii, Salvia kamelinii, Salvia karabachensis, Salvia karwinskii, Salvia keerlii, Salvia kellermanii, Salvia kermanshahensis, Salvia kiangsiensis, Salvia kiaometiensis, Salvia komarovii, Salvia korolkovii, Salvia koyamae, Salvia kronenburgii, Salvia kurdica, Salvia kuznetzovii, Salvia lachnaioclada, Salvia lachnocalyx, Salvia lachnostachys, Salvia lachnostoma, Salvia laevis, Salvia lamiifolia, Salvia lanceolata, Salvia langlassei, Salvia languidula, Salvia lanicalyx, Salvia lanicaulis, Salvia lanigera, Salvia lankongensis, Salvia lasiantha, Salvia lasiocephala, Salvia Lavandula, Salvia lavandulifolia, Salvia lavanduloides, Salvia laxispicata, Salvia lemmonii, Salvia leninae, Salvia lenta, Salvia leonia, Salvia leptophylla, Salvia leptostachys, Salvia leriifolia, Salvia leucantha, Salvia leucocephala, Salvia leucochlamys, Salvia leucodermis, Salvia leucophylla, Salvia libanensis, Salvia liguliloba, Salvia lilacinocoerulea, Salvia limbate, Salvia lineata, Salvia lipskyi, Salvia littae, Salvia lobbii, Salvia longibracteolata, Salvia longipedicellata, Salvia longispicata, Salvia longistyla, Salvia lophanthoides, Salvia loxensis, Salvia lozanii, Salvia lutescens, Salvia lycioides, Salvia lyrate, Salvia macellaria, Salvia macilenta, Salvia macrocalyx, Salvia macrochlamys, Salvia macrophylla, Salvia macrosiphon, Salvia macrostachya, Salvia madrensis, Salvia mairei, Salvia malvifolia, Salvia manantlanensis, Salvia manaurica, Salvia marashica, Salvia marci, Salvia margaritae, Salvia mattogrossensis, Salvia maximowicziana, Salvia maxonii, Salvia maymanica, Salvia mayorii, Salvia mazatlanensis, Salvia medusa, Salvia meiliensis, Salvia mekongensis, Salvia melaleuca, Salvia melissiflora, Salvia melissodora, Salvia mellifera, Salvia mentiens, Salvia merjamie, Salvia mexiae, Salvia Mexicana, Salvia microdictya, Salvia microphylla, Salvia microstegia, Salvia miltiorrhiza, Salvia minarum, Salvia miniate, Salvia mirzayanii, Salvia misella, Salvia mocinoi, Salvia modesta, Salvia modica, Salvia mohavensis, Salvia monantha, Salvia monclovensis, Salvia moniliformis, Salvia montbretia, Salvia montecristina, Salvia moorcroftiana, Salvia mornicola, Salvia mouretii, Salvia muelleri, Salvia muirii, Salvia mukerjeei, Salvia multicaulis, Salvia munzii, Salvia muscarioides, Salvia namaensis, Salvia nana, Salvia nanchuanensis, Salvia napifolia, Salvia nazalena, Salvia nemoralis, Salvia nemorosa, Salvia neovidensis, Salvia nepetoides, Salvia nervata, Salvia nervosa, Salvia nilotica, Salvia nipponica, Salvia nitida, Salvia nubicola, Salvia nubigena, Salvia nubilorum, Salvia nutans, Salvia nydeggeri, Salvia oaxacana, Salvia oblongifolia, Salvia obtorta, Salvia obtusata, Salvia obumbrate, Salvia occidentalis, Salvia occidua, Salvia occultiflora, Salvia ochrantha, Salvia ocimifolia, Salvia odontochlamys, Salvia officinalis, Salvia oligantha, Salvia oligophylla, Salvia ombrophila, Salvia omeiana, Salvia omerocalyx, Salvia opertiflora, Salvia ophiocephala, Salvia oppositiflora, Salvia orbignaei, Salvia oreopola, Salvia oresbia, Salvia orthostachys, Salvia ovalifolia, Salvia oxyphora, Salvia pachyphylla, Salvia pachypoda, Salvia pachystachya, Salvia palaestina, Salvia palealis, Salvia palifolia, Salvia pallida, Salvia palmeri, Salvia pamplonitana, Salvia pannosa, Salvia pansamalensis, Salvia paohsingensis, Salvia paposana, Salvia paraguariensis, Salvia paramicola, Salvia paramiltiorrhiza, Salvia parciflora, Salvia parryi, Salvia parvifolia, Salvia paryskii, Salvia patens, Salvia pauciflora, Salvia pauciserrata, Salvia paupercula, Salvia pavonii, Salvia penduliflora, Salvia peninsularis, Salvia pennellii, Salvia pentstemonoides, Salvia peratica, Salvia perblanda, Salvia peregrina, Salvia perlonga, Salvia perlucida, Salvia perplicata, Salvia perrieri, Salvia persepolitana, Salvia persicifolia, Salvia personata, Salvia pexa, Salvia peyronii, Salvia phaenostemma, Salvia phlomoides, Salvia piasezkii, Salvia pichinchensis, Salvia pilifera, Salvia pineticola, Salvia pinguifolia, Salvia pinnata, Salvia pisidica, Salvia platycheila, Salvia platyphylla, Salvia plebeian, Salvia plectranthoides, Salvia plumosa, Salvia plurispicata, Salvia poculata, Salvia podadena, Salvia pogonochila, Salvia polystachya, Salvia pomifera, Salvia porphyrocalyx, Salvia potaninii, Salvia potentillifolia, Salvia potus, Salvia praestans, Salvia praeterita, Salvia prasiifolia, Salvia pratensis, Salvia prattii, Salvia prilipkoana, Salvia primuliformis, Salvia pringlei, Salvia prionitis, Salvia procurrens, Salvia propinqua, Salvia prostrates, Salvia protracta, Salvia pruinose, Salvia prunelloides, Salvia prunifolia, Salvia przewalskii, Salvia pseudoincisa, Salvia pseudojaminiana, Salvia pseudomisella, Salvia pseudopallida, Salvia pseudoprivoides, Salvia pseudorosmarinus, Salvia pseudoserotina, Salvia psilantha, Salvia psilostachya, Salvia pterocalyx, Salvia pteroura, Salvia puberula, Salvia pubescens, Salvia pulchella, Salvia punctata, Salvia purpurea, Salvia purpusii, Salvia pusilla, Salvia pygmaea, Salvia qimenensis, Salvia quercetorum, Salvia quezelii, Salvia quitensis, Salvia radula, Salvia ramamoorthyana, Salvia ramose, Salvia ranzaniana, Salvia raveniana, Salvia raymondii, Salvia rechingeri, Salvia recognita, Salvia recurve, Salvia reeseana, Salvia reflexa, Salvia regla, Salvia regnelliana, Salvia reitzii, Salvia remota, Salvia repens, Salvia reptans, Salvia retinervia, Salvia reuteriana, Salvia revoluta, Salvia rhodostephana, Salvia rhombifolia, Salvia rhyacophila, Salvia rhytidea, Salvia ringens, Salvia rivularis, Salvia roborowskii, Salvia roemeriana, Salvia roscida, Salvia rosei, Salvia rosifolia, Salvia rosmarinoides, Salvia rostellata, Salvia rubescens, Salvia rubifolia, Salvia rubrifaux, Salvia rubriflora, Salvia rubropunctata, Salvia rufula, Salvia runcinate, Salvia rupicola, Salvia rusbyi, Salvia russellii, Salvia rypara, Salvia rzedowskii, Salvia saccardiana, Salvia saccifera, Salvia sacculus, Salvia sagittate, Salvia sahendica, Salvia salicifolia, Salvia samuelssonii, Salvia sanctae-luciae, Salvia santolinifolia, Salvia sapinea, Salvia sarmentosa, Salvia Saxicola, Salvia scabiosifolia, Salvia scabra, Salvia scabrata, Salvia scabrida, Salvia scandens, Salvia scapiformis, Salvia scapose, Salvia schimperi, Salvia schizocalyx, Salvia schizochila, Salvia schlechteri, Salvia schmalbausenii, Salvia sciaphila, Salvia sclarea, Salvia sclareoides, Salvia sclareopsis, Salvia scoparia, Salvia scutellarioides, Salvia scytinophylla, Salvia secunda, Salvia seemannii, Salvia segtinophylla, Salvia selguapensis, Salvia selleana, Salvia sellowiana, Salvia semiatrata, Salvia seravschanica, Salvia sericeotomentosa, Salvia serotina, Salvia serpyllifolia, Salvia serranoae, Salvia sessei, Salvia sessilifolia, Salvia setosa, Salvia setulose, Salvia shannonii, Salvia sharifii, Salvia sharpie, Salvia sibthorpii, Salvia sigchosica, Salvia sikkimensis, Salvia silvarum, Salvia similis, Salvia sinaloensis, Salvia sinica, Salvia smithii, Salvia smyrnaea, Salvia somalensis, Salvia sonchifolia, Salvia sonklarii, Salvia sonomensis, Salvia sophrona, Salvia sordida, Salvia sparsiflora, Salvia spathacea, Salvia speciosa, Salvia speirematoides, Salvia sphacelifolia, Salvia sphacelioides, Salvia spinosa, Salvia splendens, Salvia sprucei, Salvia squalens, Salvia stachydifolia, Salvia stachyoides, Salvia staminea, Salvia stenophylla, Salvia stibalii, Salvia stolonifera, Salvia striata, Salvia strobilanthoidea, Salvia styphelos, Salvia subaequalis, Salvia subglabra, Salvia subhastata, Salvia subincisa, Salvia submutica, Salvia subobscura, Salvia subpalmatinervis, Salvia subpatens, Salvia subrotunda, Salvia subrubens, Salvia subscandens, Salvia substolonifera, Salvia sucrensis, Salvia suffruticosa, Salvia summa, Salvia superba, Salvia synodonta, Salvia syriaca, Salvia tafallae, Salvia taraxacifolia, Salvia tchihatcheffii, Salvia tebesana, Salvia teddii, Salvia Tehuacana, Salvia tenella, Salvia tenuiflora, Salvia tepicensis, Salvia teresae, Salvia tesquicola, Salvia tetrodonta, Salvia texana, Salvia thermarum, Salvia thomasiana, Salvia thormannii, Salvia thymoides, Salvia thyrsiflora, Salvia tianschanica, Salvia tigrine, Salvia tiliifolia, Salvia tingitana, Salvia tobeyi, Salvia tolimensis, Salvia tomentella, Salvia tomentosa, Salvia tonalensis, Salvia topiensis, Salvia tortuensis, Salvia tortuosa, Salvia townsendii, Salvia trachyphylla, Salvia transhimalaica, Salvia transsylvanica, Salvia trautvetteri, Salvia triangularis, Salvia trichocalycina, Salvia trichoclada, Salvia trichopes, Salvia trichostephana, Salvia tricuspidate, Salvia tricuspis, Salvia trifilis, Salvia trijuga, Salvia tubifera, Salvia tubiflora, Salvia tubulosa, Salvia tuerckheimii, Salvia turcomanica, Salvia turdi, Salvia turneri, Salvia tuxtlensis, Salvia tysonii, Salvia uliginosa, Salvia umbratical, Salvia umbraticola, Salvia umbratilis, Salvia uncinate, Salvia unguella, Salvia unicostate, Salvia univerticillata, Salvia uribei, Salvia urica, Salvia urmiensis, Salvia urolepis, Salvia urticifolia, Salvia uruapana, Salvia valentina, Salvia vargasii, Salvia vaseyi, Salvia vasta, Salvia veneris, Salvia venulosa, Salvia verapazana, Salvia verbascifolia, Salvia verbenaca, Salvia verecunda, Salvia vergeduzica, Salvia vermifolia, Salvia veronicifolia, Salvia verticillate, Salvia vestita, Salvia vialis, Salvia villosa, Salvia virgata, Salvia viridis, Salvia viscida, Salvia viscidifolia, Salvia viscosa, Salvia vitifolia, Salvia vvedenskii, Salvia wagneriana, Salvia wardii, Salvia warszewicziana, Salvia weberbaueri, Salvia weihaiensis, Salvia wendelboi, Salvia whitefoordiae, Salvia whitehousei, Salvia wiedemannii, Salvia willeana, Salvia xalapensis, Salvia xanthocheila, Salvia xanthophylla, Salvia xanthotricha, Salvia xeropapillosa, Salvia yosgadensis, Salvia yukoyukparum, Salvia yunnanensis, and Salvia zacualpanensis.

In some embodiments, the Salvia extract is an extract of Salvia officinalis. In other embodiments, the Salvia extract is an extract of Salvia officinalis leaves.

The genus Magnolia is a group of flowering plants in the family Magnoliaceae, which includes, inter alia, the species Magnolia acuminata, Magnolia alba, Magnolia albosericea, Magnolia allenii, Magnolia amazonica, Magnolia amoena, Magnolia angatensis, Magnolia angustioblonga, Magnolia annamensis, Magnolia arcabucoana, Magnolia argyrothricha, Magnolia aromatica, Magnolia ashtonii, Magnolia baillonii, Magnolia balansae, Magnolia banghamii, Magnolia bankardiorum, Magnolia bawangensis, Magnolia betongensis, Magnolia bintuluensis, Magnolia biondii, Magnolia blaoensis, Magnolia blumei, Magnolia boliviana, Magnolia borneensis, Magnolia braianensis, Magnolia calimaensis, Magnolia calophylla, Magnolia calophylloides, Magnolia campbellii, Magnolia cararensis, Magnolia caricifragrans, Magnolia carsonii, Magnolia cathcartii, Magnolia cavaleriei, Magnolia caveana, Magnolia cespedesii, Magnolia champaca, Magnolia championii, Magnolia changhuntana, Magnolia chapensis, Magnolia chevalieri, Magnolia chimantensis, Magnolia chocoensis, Magnolia citrata, Magnolia clemensiorum, Magnolia coco, Magnolia colombiana, Magnolia compressa, Magnolia conifera, Magnolia coriacea, Magnolia coronata, Magnolia crassipes, Magnolia cristalensis, Magnolia cubensis, Magnolia cylindrica, Magnolia dandyi, Magnolia dawsoniana, Magnolia decidua, Magnolia delavayi, Magnolia denudate, Magnolia dixonii, Magnolia dodecapetala, Magnolia doltsopa, Magnolia domingensis, Magnolia duclouxii, Magnolia duperreana, Magnolia ekmannii, Magnolia elegans, Magnolia elegantifolia, Magnolia emarginata, Magnolia ernestii, Magnolia espinalii, Magnolia figlarii, Magnolia figo, Magnolia fistulosa, Magnolia flaviflora, Magnolia floribunda, Magnolia fordiana, Magnolia foveolate, Magnolia fraseri, Magnolia fujianensis, Magnolia fulva, Magnolia garrettii, Magnolia georgii, Magnolia gigantifolia, Magnolia gilbertoi, Magnolia gioi, Magnolia globosa, Magnolia gloriensis, Magnolia grandiflora, Magnolia grandis, Magnolia griffithii, Magnolia guangdongensis, Magnolia guangxiensis, Magnolia guatapensis, Magnolia guatemalensis, Magnolia guatemalensis, Magnolia guerrerensis, Magnolia gustavii, Magnolia hamorii, Magnolia henaoi, Magnolia henryi, Magnolia hernandezii, Magnolia hodgsonii, Magnolia hongheensis, Magnolia hookeri, Magnolia hypolampra, Magnolia iltisiana, Magnolia insignis, Magnolia irwiniana, Magnolia iteophylla, Magnolia jaenensis, Magnolia jardinensis, Magnolia kachirachirai, Magnolia katiorum, Magnolia kingii, Magnolia kisopa, Magnolia kobus, Magnolia koordersiana, Magnolia krusei, Magnolia kwangsiensis, Magnolia kwangtungensis, Magnolia lacandonica, Magnolia lacei, Magnolia laevifolia, Magnolia lanuginosoides, Magnolia lasia, Magnolia lanuginosa, Magnolia lenticellata, Magnolia leveilleana, Magnolia liliifera, Magnolia liliiflora, Magnolia loebneri, Magnolia longipedunculata, Magnolia lotungensis, Magnolia lucida, Magnolia macclurei, Magnolia macklottii, Magnolia macrophylla, Magnolia magnifolia, Magnolia mahechae, Magnolia manguillo, Magnolia mannii, Magnolia mariusjacobsia, Magnolia martini, Magnolia masticata, Magnolia maudiae, Magnolia mediocris, Magnolia Mexicana, Magnolia microcarpa, Magnolia microtricha, Magnolia minor, Magnolia montana, Magnolia morii, Magnolia nana, Magnolia narinensis, Magnolia neillii, Magnolia nilagirica, Magnolia nitida, Magnolia oblonga, Magnolia obovalifolia, Magnolia obovata, Magnolia odora, Magnolia odoratissima, Magnolia officinalis, Magnolia omeiensis, Magnolia opipara, Magnolia ovata, Magnolia ovoidea, Magnolia pacifica, Magnolia pahangensis, Magnolia pallescens, Magnolia panamensis, Magnolia patungensis, Magnolia persuaveolens, Magnolia philippinensis, Magnolia pealiana, Magnolia platyphylla, Magnolia pleiocarpa, Magnolia poasana, Magnolia polyhypsophylla, Magnolia portoricensis, Magnolia praecalva, Magnolia ptaritepuiana, Magnolia pterocarpan, Magnolia pubescens, Magnolia punduana, Magnolia quetzal, Magnolia rabaniana, Magnolia rajaniana, Magnolia rimachii, Magnolia rostrata, Magnolia rufibarbata, Magnolia sabahensis, Magnolia salicifolia, Magnolia sapaensis, Magnolia sarawakensis, Magnolia sambuensis, Magnolia santanderiana, Magnolia sargentiana, Magnolia schiedeana, Magnolia scortechinii, Magnolia sellowiana, Magnolia sharpie, Magnolia shiluensis, Magnolia sieboldii, Magnolia silvioi, Magnolia singapurensis, Magnolia sinica, Magnolia sirindhorniae, Magnolia sororum, Magnolia soulangeana, Magnolia sphaerantha, Magnolia splendens, Magnolia sprengeri, Magnolia stellata, Magnolia striatifolia, Magnolia subulifera, Magnolia sulawesiana, Magnolia sumatrae, Magnolia tamaulipana, Magnolia thailandica, Magnolia tripetala, Magnolia tsiampacca, Magnolia urraoense, Magnolia utilis, Magnolia vazquezii, Magnolia venezuelensis, Magnolia ventii, Magnolia villosa, Magnolia virginiana, Magnolia virolinensis, Magnolia vrieseana, Magnolia wilsonii, Magnolia wolfii, Magnolia xanthantha, Magnolia xiana, Magnolia xinganensis, Magnolia yarumalense, Magnolia yoroconte, Magnolia yunnanensis, Magnolia yuyuanensis, Magnolia zenii, and Magnolia zhengyiana.

In some embodiments, the Magnolia extract is an extract of Magnolia officinalis. In other embodiments, the Magnolia extract is an extract of Magnolia officinalis bark.

The genus Commiphora is a genus of flowering plants in the family Burseraceae, which includes, inter alia, the species Commiphora africana, Commiphora alaticaulis, Commiphora angolensis, Commiphora boranensis, Commiphora caudata, Commiphora ciliata, Commiphora confusa, Commiphora corrugata, Commiphora erosa, Commiphora gileadensis, Commiphora glandulosa, Commiphora guidottii, Commiphora guillauminii, Commiphora habessinica, Commiphora harveyi, Commiphora humbertii, Commiphora kataf, Commiphora kua, Commiphora madagascariensis, Commiphora monoica, Commiphora mossambicensis, Commiphora myrrha, Commiphora saxicola, Commiphora schimperi, Commiphora simplicifolia, Commiphora sphaerocarpa, Commiphora stocksiana, Commiphora unilobata, and Commiphora wightii.

In some embodiments, the Commiphora extract is an extract of Commiphora myrrha. In other embodiments, the Commiphora extract is an extract of Commiphora myrrha resin.

The genus Boswellia is another genus of flowering plants in the family Burseraceae, which includes, inter alia, the species Boswellia ameero, Boswellia boranensis, Boswellia bricchettii, Boswellia bullata, Boswellia chariensis, Boswellia dalzielii, Boswellia dioscoridis, Boswellia elegans, Boswellia elongate, Boswellia frereana, Boswellia globosa, Boswellia hildebrandtii, Boswellia holstii, Boswellia madagascariensis, Boswellia microphylla, Boswellia multifoliolate, Boswellia nana, Boswellia neglecta, Boswellia odorata, Boswellia ogadensis, Boswellia ovalifoliolata, Boswellia papyrifera, Boswellia pirottae, Boswellia popoviana, Boswellia rivae, Boswellia ruspoliana, Boswellia sacra, Boswellia serrata, and Boswellia socotrana.

In some embodiments, the Boswellia extract is an extract of Boswellia serrata. In other embodiments, the Boswellia extract is an extract of Boswellia serrata resin.

The genus Dipterocarpus is another genus of plants in the family Dipterocarpaceae, which includes, inter alia, the species Dipterocarpus acutangulus, Dipterocarpus alatus, Dipterocarpus applanatus, Dipterocarpus baudii, Dipterocarpus borneensis, Dipterocarpus bourdilloni, Dipterocarpus caudatus, Dipterocarpus caudiferus, Dipterocarpus chartaceus, Dipterocarpus cinereus, Dipterocarpus concavus, Dipterocarpus condorensis, Dipterocarpus confertus, Dipterocarpus conformis, Dipterocarpus coriaceus, Dipterocarpus cornutus, Dipterocarpus costatus, Dipterocarpus costulatus, Dipterocarpus crinitus, Dipterocarpus cuspidatus, Dipterocarpus dyeri, Dipterocarpus elongatus, Dipterocarpus eurynchus, Dipterocarpus fagineus, Dipterocarpus fusiformis, Dipterocarpus geniculatus, Dipterocarpus glabrigemmatus, Dipterocarpus glandulosus, Dipterocarpus globosus, Dipterocarpus gracilis, Dipterocarpus grandiflorus, Dipterocarpus hasseltii, Dipterocarpus hispidus, Dipterocarpus humeratus, Dipterocarpus indicus, Dipterocarpus insignis, Dipterocarpus intricatus, Dipterocarpus kerrii, Dipterocarpus kunstleri, Dipterocarpus lamellatus, Dipterocarpus littoralis, Dipterocarpus lowii, Dipterocarpus megacarpus, Dipterocarpus mundus, Dipterocarpus nudus, Dipterocarpus oblongifolius, Dipterocarpus obtusifolius, Dipterocarpus ochraceus, Dipterocarpus orbicularis, Dipterocarpus pachyphyllus, Dipterocarpus palembanicus, Dipterocarpus perakensis, Dipterocarpud pseudocornutus, Dipterocarpus retusus, Dipterocarpus rigidus, Dipterocarpus rotundifolius, Dipterocarpus sarawakensis, Dipterocarpus semivestitus, Dipterocarpus stellatus, Dipterocarpus sublamellatus, Dipterocarpus tempehes, Dipterocarpus tuberculatus, Dipterocarpus turbinatus, Dipterocarpus Validus, Dipterocarpus verrucosus, and Dipterocarpus zeylanicus.

In some embodiments, the Dipterocarpus extract is an extract of Dipterocarpus turbinatus.

In other embodiments, the Dipterocarpus extract is an extract of Dipterocarpus turbinatus resin.

In some embodiments, the Copaiba extract is an extract of Copaiba langsdorffii. In other embodiments, the Dipterocarpus extract is an extract of Copaiba langsdorffii resin or bark.

The genus Malpighia is a genus of flowering plants in the family Malpighiaceae, which includes, inter alia, the species Malpighia aquifolia, Malpighia cauliflora, Malpighia coccigera, Malpighia cubensis, Malpighia emarginata, Malpighia fucata, Malpighia glabra, Malpighia harrisii, Malpighia mexicana, Malipighia obtusifolia, Malpighia polytricha, Malpighia proctorii, Malpighia selosa, Malpighia suberosa, Malpighia urens.

In some embodiments, the Malpighia extract is an extract of Malpighia glabra or Malpighia emarginata. In other embodiments, the Malpighia extract is an extract of Malpighia glabra or Malpighia emarginata fruit.

The genus Thymus is a group of plants in the family Lamiaceae, which includes, inter alia, the species Thymus adamovicii, Thymus altaicus, Thymus amurensis, Thymus boissieri, Thymus bracteosus, Thymus broussonetii, Thymus caespititius, Thymus camphoratus, Thymus capitatus, Thymus capitellatus, Thymus camphoratus, Thymus carnosus, Thymus cephalotus, Thymus cherlerioides, Thymus ciliatus, Thymus cilicicus, Thymus cimicinus, Thymus citriodorus, Thymus comosus, Thymus comptus, Thymus curtus, Thymus decussatus, Thymus disjunctus, Thymus doerfleri, Thymus glabrescens, Thymus herba-barona, Thymus hirsutus, Thymus hyemalis, Thymus inaequalis, Thymus integer, Thymus lanuginosus, Thymus leucospermus, Thymus leucotrichus, Thymus longicaulis, Thymus longiflorus, Thymus mandschuricus, Thymus marschallianus, Thymus mastichina, Thymus membranaceus, Thymus mongolicus, Thymus moroderi, Thymus nervulosus, Thymus nummularis, Thymus odoratissimus, Thymus pallasianus, Thymus pallidus, Thymus pannonicus, Thymus praecox, Thymus proximus, Thymus pseudolanuginosus, Thymus pulegioides, Thymus quinquecostatus, Thymus richardii, Thymus satureioides, Thymus serpyllum, Thymus sibthorpii, Thymus striatus, Thymus thracicus, Thymus villosus, Thymus vulgaris and Thymus zygis.

In some embodiments, the Thymus extract is an extract of Thymus vulgaris. In other embodiments, the Thymus extract is an extract of Thymus vulgaris leaves.

The genus Origanum is another herbaceous perennials group of plants in the family Lamiaceae, which includes, inter alia, the species Origanum acutidens, Origanum adanense, Origanum adonidis, Origanum akhdarense, Origanum amanum, Origanum barbarae, Origanum bargyli, Origanum bilgeri, Origanum boissieri, Origanum calcaratum, Origanum compactum, Origanum cordifolium, Origanum cyrenaicum, Origanum dayi, Origanum Dictamnus, Origanum dolichosiphon, Origanum ehrenbergii, Origanum elongatum, Origanum floribundum, Origanum haradjanii, Origanum haussknechtii, Origanum husnucan-baseri, Origanum hypericifolium, Origanum intercedens, Origanum intermedium, Origanum isthmicum, Origanum jordanicum, Origanum laevigatum, Origanum leptocladum, Origanum libanoticum, Origanum majorana, Origanum lirium, Origanum majoricum, Origanum microphyllum, Origanum minoanum, Origanum minutiflorum, Origanum munzurense, Origanum nebrodense, Origanum onites, Origanum pabotii, Origanum pampaninii, Origanum petraeum, Origanum punonense, Origanum ramonense, Origanum rotundifolium, Origanum saccatum, Origanum scabrum, Origanum sipyleum, Origanum solymicum, Origanum symes, Origanum syriacum, Origanum vetteri, Origanum vogelii, and Origanum vulgare.

In some embodiments, the Origanum extract is an extract of Origanum vulgare. In other embodiments, the Origanum extract is an extract of Origanum vulgare leaves.

The genus Cymbopogon is a group of plants in the family Poaceae, which includes, inter alia, the species Cymbopogon ambiguous, Cymbopogon annamensis, Cymbopogon bhutanicus, Cymbopogon bombycinus, Cymbopogon caesius, Cymbopogon calcicole, Cymbopogon calciphilus, Cymbopogon cambogiensis, Cymbopogon citratus, Cymbopogon clandestinus, Cymbopogon coloratus, Cymbopogon commutatus, Cymbopogon densiflorus, Cymbopogon dependens, Cymbopogon dieterlenii, Cymbopogon distans, Cymbopogon exsertus, Cymbopogon flexuosus, Cymbopogon gidarba, Cymbopogon giganteus, Cymbopogon globosus, Cymbopogon goeringii, Cymbopogon gratus, Cymbopogon jwarancusa, Cymbopogon khasianus, Cymbopogon liangshanensis, Cymbopogon mandalaiaensis, Cymbopogon marginatus, Cymbopogon martini, Cymbopogon mekongensis, Cymbopogon microstachys, Cymbopogon microthecus, Cymbopogon minor, Cymbopogon minutiflorus, Cymbopogon nardus, Cymbopogon nervatus, Cymbopogon obtectus, Cymbopogon osmastonii, Cymbopogon pendulus, Cymbopogon polyneuros, Cymbopogon pospischilii, Cymbopogon procerus, Cymbopogon pruinosus, Cymbopogon queenslandicus, Cymbopogon quinhonensis, Cymbopogon rectus, Cymbopogon refractus, Cymbopogon schoenanthus, Cymbopogon tortilis, Cymbopogon tungmaiensis, Cymbopogon winterianus, and Cymbopogon xichangensis.

In some embodiments, the Cymbopogon extract is an extract of Cymbopogon citratus. In other embodiments, the Anethum extract is an extract of Cymbopogon citratus leaves.

In some embodiments, the Anethum extract is an extract of Anethum graveolens. In other embodiments, the Anethum extract is an extract of Anethum graveolens seeds.

The genus Syzygium is a group of flowering plants in the family Myrtaceae, which includes, inter alia, the species Syzygium alliiligneum, Syzygium amplifolium, Syzygium andamanicum, Syzygium anisatum, Syzygium anisosepalum, Syzygium angophoroides, Syzygium antisepticum, Syzygium aqueum, Syzygium aromaticum, Syzygium austral, Syzygium beddomei, Syzygium bourdillonii, Syzygium canicortex, Syzygium caryophyllatum, Syzygium chanlos, Syzygium chavaran, Syzygium cinereum, Syzygium conglomeratum, Syzygium contractum, Syzygium cordatum, Syzygium cordifolium, Syzygium cormiflorum, Syzygium corynanthum, Syzygium corynocarpa, Syzygium courtallense, Syzygium crebrinerve, Syzygium cumini, Syzygium curranii, Syzygium densiflorum, Syzygium diffusum, Syzygium discophorum, Syzygium duthieanum, Syzygium dyerianum, Syzygium elegans, Syzygium erythrocalyx, Syzygium eucalyptoides, Syzygium fergusonii, Syzygium fibrosum, Syzygium fijiense, Syzygium flosculiferum, Syzygium forte, Syzygium formosanum, Syzygium francisii, Syzygium fullagarii, Syzygium glaucum, Syzygium gracilipes, Syzygium graeme-andersoniae, Syzygium grande, Syzygium guehoi, Syzygium gustavioides, Syzygium guineense, Syzygium hodgkinsoniae, Syzygium jambos, Syzygium jasminifolium, Syzygium kemamanense, Syzygium kiahii, Syzygium koordersianum, Syzygium kuranda, Syzygium leucoxylon, Syzygium luehmannii, Syzygium maire, Syzygium makul, Syzygium malaccense, Syzygium manii, Syzygium maingayi, Syzygium megacarpum, Syzygium micranthum, Syzygium microphyllum, Syzygium minus, Syzygium monimioides, Syzygium moorei, Syzygium myhendrae, Syzygium neesianum, Syzygium nemestrinum, Syzygium nervosum, Syzygium oblatum, Syzygium occidentale, Syzygium oleosum, Syzygium oliganthum, Syzygium oreophilum, Syzygium palghatense, Syzygium paniculatum, Syzygium parameswaranii, Syzygium patentinerve, Syzygium papyraceum, Syzygium pauper, Syzygium pendens, Syzygium perakense, Syzygium pergamentaceum, Syzygium phaeophyllum, Syzygium politum, Syzygium polyanthum, Syzygium pondoense, Syzygium praineanum, Syzygium pseudofastigiatum, Syzygium purpureum, Syzygium quadrangulatum, Syzygium quadribracteatum, Syzygium ramavarmae, Syzygium revolutum, Syzygium richii, Syzygium samarangense, Syzygium sandwicensis, Syzygium smithii, Syzygium suborbiculare, Syzygium syzygioides, and Syzygium travancoricum.

In some embodiments, the Syzygium extract is an extract of Syzygium aromaticum. In other embodiments, the Syzygium extract is an extract of Syzygium aromaticum flowers or buds.

The genus Nigella is a genus of annual plants in the family Ranunculaceae, which includes, inter alia, the species Nigella arvensis, Nigella carpatha, Nigella damascena, Nigella degenii, Nigella deserti, Nigella doerfleri, Nigella elata, Nigella fumariifola, Nigella hispanica, Nigella latisecta, Nigella nigellastrum, Nigella orientalis, Nigella oxypetala, Nigella papillosa, Nigella sativa, Nigella segetalis, Nigella stricta and Nigella unguicularis.

In some embodiments, the Nigella extract is an extract of Nigella sativa. In other embodiments, the Nigella extract is an extract of Nigella sativa seeds.

The genus Pimenta is a group of flowering plants in the myrtle family, which includes, inter alia, the species Pimenta adenoclada, Pimenta berciliae, Pimenta cainitoides, Pimenta dioica (allspice), Pimenta ferruginea, Pimenta filipes, Pimenta guatemalensis, Pimenta haitiensis, Pimenta intermedia, Pimenta jamaicensis, Pimenta obscura, Pimenta odiolens, Pimenta oligantha, Pimenta podocarpoides, Pimenta pseudocaryophyllus, Pimenta racemosa, Pimenta samanensis, Pimenta richardii, and Pimenta yumana.

In some embodiments, the Pimenta extract is an extract of Pimenta dioica. In other embodiments, the Pimenta extract is an extract of Pimenta dioica fruit.

According to some embodiments, the additional plant extract may be selected from wherein said at least one additional plant source is selected from Cinnamomum cassia extract, Paeonia lactiflora extract, Salvia officinalis extract, Magnolia officinalis extract, Commiphora myrrha extract, Boswellia serrata extract, Dipterocarpus turbinatus extract, Copaiba langsdorffii extract, Malpighia emarginata extract, Malpighia glabra extract, Thymus vulgaris extract, Origanum vulgare extract, Cymbopogon citratus extract, Anethum graveolens extract, Syzygium aromaticum extract, Nigella sativa extract, Pimenta dioica extract, maltol extracted from a plant source and mixtures thereof.

According to some other embodiments, the additional plant extract can be selected Cinnamomum cassia bark or twig extract, Paeonia lactiflora roots extract, Salvia officinalis leaves extract, Magnolia officinalis bark extract, Commiphora myrrha resin extract, Boswellia serrata resin extract, Dipterocarpus turbinatus resin extract, Copaiba langsdorffii resin or bark extract, Malpighia glabra or Malpighia emarginata fruit extract, Thymus vulgaris leaves extract, Origanum vulgare leaves extract, Cymbopogon citratus leaves extract, Anethum graveolens seeds extract, Syzygium aromaticum flowers or buds extract, Nigella sativa seeds extract, Pimenta dioica fruit extract, maltol and mixtures thereof.

Another aspect of this disclosure provides antimicrobial composition comprising at antimicrobial effective amount of a mixture of agents, the mixture of agents consisting of at least one saponin extract, at least one plant extract from at least one plant species selected from the genus Styrax, Myroxylon and/or Myrocarpus and mixtures thereof, and at least one additional plant extract from an additional plant source, the saponin extract being from a saponin source different from said at least one plant species and from said additional plant source.

In another aspect, there is provided an antimicrobial composition comprising at antimicrobial effective amount of a mixture of agents, the mixture of agents consisting of: a saponin extract selected from Camellia oleifera extract, Camellia sinensis extract, Quillaja saponaria extract, Sapindus mukorossi extract, Sapindus saponaria extract, and Saponaria officinalis extract, and any mixture thereof; a Styrax extract from Styrax paralleloneurus (Sumatra benzoin), Styrax tonkinensis (Siam Benzoin), Styrax formosanus, Styrax peruvianum, Styrax tolu and mixtures thereof; and at least one plant extract from an additional plant source selected from Cinnamomum cassia, Paeonia Lactiflora, Salvia Officinalis, Magnolia Officinalis, Commiphora myrrha, Boswellia serrata, Dipterocarpus turbinatus, Copaiba langsdorffii, Malpighia emarginata, Malpighia glabra, Thymus vulgaris, Origanum vulgare, Cymbopogon citratus, Anethum graveolens, Syzygium aromaticum, Nigella sativa, Pimenta dioica, and maltol extracted from a plant source; and optionally at least one additive.

Yet a further aspect provides for an add-on composition consisting of a combination of at least one saponin extract, at least one plant extract from at least one plant species selected from the genus Styrax, Myroxylon and/or Myrocarpus and mixtures thereof, and at least one additional plant extract from an additional plant source, the saponin extract being from a saponin source different from said at least one plant species and from said additional plant source.

The term add-on composition (or add-on formulation) is meant to refer to a composition that is added to another, already-prepared composition. For example, the add-on composition may be added to a variety of other products, e.g. shampoo, soap, cream, lotion, etc., to provide these products with a desired property. Such property may be antimicrobial activity, foaming, viscosity modification, improved absorbance, and the like. It is of note that the add-on formulation may be formulated separately from the composition of the product, and then added to the product. However, it is to be understood that each of the components of the add-on formulation may be individually added to the composition of the product at any desired addition sequence.

The compositions of this disclosure can be prepared by any commonly used method for preparing a composition of materials. For example, the components of the compositions may be added as solids and mixed together, or one of the components may be added to the other in the form of a solution which may, if desired be evaporated or lyophilized after mixing for obtaining a homogeneous solution.

In another aspect, there is provided an antimicrobial composition selected from:
(a) a composition comprising saponin extract, Styrax extract and maltol;
(b) a composition comprising saponin extract, Styrax extract, maltol, and citric acid;
(c) a composition comprising saponin extract, Styrax extract and Cinnamomum extract;
(d) a composition comprising saponin extract, Styrax extract, Cinnamomum extract and maltol;
(e) a composition comprising saponin extract, Styrax extract, and Magnolia extract;
(f) a composition comprising saponin extract, Styrax extract, and Paeonia extract;
(g) a composition comprising saponin extract, Styrax extract, Cinnamomum extract, citric acid and maltol;
(h) a composition comprising saponin extract, Styrax extract, Cinnamomum extract, and citric acid;
(i) a composition comprising saponin extract, Styrax extract, Cinnamomum extract, Paeonia extract, citric acid and maltol;
(j) a composition comprising saponin extract, Styrax extract, Cinnamomum extract, and Salvia extract;
(k) a composition comprising saponin extract, Styrax extract, Cinnamomum cassia extract, and shikimic acid;
(l) a composition comprising saponin extract, Styrax extract, and Nigella sativa extract;
(m) a composition comprising saponin extract, Styrax extract, and Pimenta dioica extract;
(n) a composition comprising saponin extract, Styrax extract, and Cymbopogon citrus extract;
(o) a composition comprising saponin extract, Styrax extract, and Salvia officinalis leaves extract;
(p) a composition comprising saponin extract, Styrax extract, and Malpighia emarginata extract
(q) a composition comprising saponin extract, Styrax extract, and Thymus vulgaris extract;
(r) a composition comprising saponin extract, Styrax extract, and Origanum vulgare extract; and
(s) a composition comprising saponin extract, Styrax extract, and Syzygium aromaticum extract.

By another aspect, there is provided an antimicrobial composition selected from:
(a) a composition comprising saponin extract, Styrax extract and maltol;
(b) a composition comprising saponin extract, Styrax extract, maltol, and citric acid;
(c) a composition comprising saponin extract, Styrax extract and Cinnamomum cassia extract;
(d) a composition comprising saponin extract, Styrax extract, Cinnamomum cassia extract and maltol;
(e) a composition comprising saponin extract, Styrax extract, and Magnolia Officinalis extract;
(f) a composition comprising saponin extract, Styrax extract, and Paeonia Lactiflora extract;
(g) a composition comprising saponin extract, Styrax extract, Cinnamomum cassia extract, citric acid and maltol;
(h) a composition comprising saponin extract, Styrax extract, Cinnamomum cassia extract, and citric acid;
(i) a composition comprising saponin extract, Styrax extract, Cinnamomum cassia extract, Paeonia Lactiflora extract, citric acid and maltol;
(j) a composition comprising saponin extract, Styrax extract, Cinnamomum cassia extract, and Salvia officinalis extract; and
(k) a composition comprising saponin extract, Styrax extract, Cinnamomum cassia extract, and shikimic acid.

According to some embodiments, the composition comprises at least 0.04 wt % maltol, at least 0.075 wt % Sapindus mukorossi extract, and 0.025 wt % Styrax extract.

According to other embodiments, the composition comprises at least 0.04 wt % maltol, at least 0.075 wt % Sapindus mukorossi extract, and at least 0.003 wt % citric acid.

According to some other embodiments, the composition comprises at least 0.02 wt % maltol, at least 0.0375 wt % Sapindus mukorossi extract, 0.0125 wt % Styrax extract, and at least 0.0015 wt % citric acid.

As will be further demonstrated below, the compositions of this disclosure exhibit antimicrobial properties which render the compositions suitable for a variety of applications in the fields of, e.g., cosmetics, therapeutics, foodstuffs and as material preservation.

The composition of this disclosure may thus be formulated into a variety of formulations, such as a cosmetic formulation, a therapeutic formulation, an antimicrobial formulation, a food additive formulation and a preservative formulation. Each of the aforementioned formulations may further comprise an excipient, diluents, or carrier suitable for the particular application, together with at least one additional additive as disclosed herein.

In another of its aspects, the disclosure provides a cosmetic or cleansing formulation comprising compositions of the present disclosure as defined in the various embodiments hereinabove.

The cosmetic/cleansing formulations are typically formulated in a form adapted for topical application comprising a cosmetically or dermatologically acceptable medium, namely a medium which is suitable for application onto the skin of a subject (human or non-human). The medium may be in the form of aqueous or hydroalcoholic solution, an oil-in-water or water-in-oil emulsion, a microemulsion, aqueous or anhydrous gels, serum, or else a dispersion of vesicles, a patch, cream, spray, salve, ointment, lotion, gel, solution, suspension, or any other known cosmetically acceptable form. The formulation may alternatively be formulated for application to the human skin, hair, eyelashes, eyebrows, or nails.

In addition, the formulation may contain other standard additives such as an emollient, moisturizer, thickener, emulsifier, neutralizer, coloring agent, a fragrance, absorber or filter, preservative and/or gelling agent such as those described below, filler such as nylon, a sunscreen agent, electrolytes, proteins, antioxidants and chelating agents.

The formulation may also further comprise at least one active ingredient such as peptide active ingredients, vegetable extracts, anti-age agents, anti-wrinkle agents, soothing agents, radical scavengers, UV absorbing agents, agents stimulating the synthesis of dermal macromolecules or the energy metabolism, hydrating agents, anti-bacterial agents, anti-fungal agents, anti-inflammatory agents, anesthetic agents, agents modulating cutaneous differentiation, pigmentation or de-pigmentation, agents stimulating nail or hair growth.

In some embodiments, each of the aforementioned additives/active ingredients is generally present in an amount of between about 0.1 and 30 wt % of the total weight of the formulation.

Suitable emollients for use in a cosmetic/cleansing formulation include, for example, optionally hydroxy-substituted C₈-C₅₀ unsaturated fatty acids and esters thereof, C₁-C₂₄ esters of C₈-C₃₀ saturated fatty acids such as isopropyl myristate, cetyl palmitate and octyldodecylmyristate (Wickenol 142), beeswax, saturated and unsaturated fatty alcohols such as behenyl alcohol and cetyl alcohol, hydrocarbons such as mineral oils, petrolatum, squalane, fatty sorbitan esters, lanolin and lanolin derivatives, such as lanolin alcohol ethoxylated, hydroxylated and acetylated lanolins, cholesterol and derivatives thereof, animal and vegetable triglycerides such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppy seed oil, pine oil, castor oil, soybean oil, avocado oil, safflower oil, coconut oil, hazelnut oil, olive oil, grape seed oil, and sunflower seed oil and C₁-C₂₄ esters of dimer and trimer acids such as diisopropyl dimerate, diisostearylmalate, diisostearyldimerate and triisostearyltrimerate.

In some embodiments, the emollients used in a formulation include isocetyl alcohol, octyl palmitate, isostearyl neopentanoate and isocetyl stearyl stearate, natural or synthetic oils selected from mineral, vegetable, and animal oils, fats and waxes, fatty acid esters, fatty alcohols, alkylene glycol and polyalkylene glycol ethers and esters, fatty acids and mixtures thereof.

The emollients may be used independently or in mixtures and may be present in the composition of the present disclosure in an amount from about 1 to about 98% by weight, and in some embodiments are present in an amount from about 5% to about 15% by weight of the total formulation.

Suitable emulsifiers for use in a cosmetic/cleansing formulation according to the present disclosure include glyceryl stearate and laureth 23, PEG 20 stearate, and mink-amidopropyl dimethyl 2-hydroxyethylammonium chloride.

Typical moisturizers are glycerin, petrolatum and maleated vegetable oil.

The formulation may also contain a hydrophilic gelling agent, which may be selected from water-soluble or colloidal water-soluble polymers, such as cellulose ethers (e.g. hydroxyethyl cellulose, methyl cellulose, hydroxypropylmethyl cellulose), polyvinylalcohol, polyquaternium-10, guar gum, hydroxypropyl guar gum, xanthan gum, Aloe vera gel, amla, carrageenan, oat flour, starch (from corn rice or other plants), gelatin (porcine skin), ghatty gum, gum Arabic, inulin (from chicory), Konjac gum, locust bean gum, marshmallow root, pectin, quinoa extract, red alga, solagum and tragacanth gum (TG).

In further embodiments, the hydrophilic gelling agents are selected amongst acrylic acid/ethyl acrylate copolymers and the carboxyvinyl polymers sold by the B.F. Goodrich Company under the trademark of Carbopol resins. These resins consist essentially of a colloidal water-soluble polyalkenyl polyether crosslinked polymer of acrylic acid crosslinked with from 0.75% to 2.00% of a crosslinking agent such as polyallyl sucrose or polyallyl pentaerythritol. Examples include Carbopol 934, Carbopol 940, Carbopol 950, Carbopol 980, Carbopol 951 and Carbopol 981. Carbopol 934 is a water-soluble polymer of acrylic acid crosslinked with about 1 of polyallyl ether of sucrose having an average of about 5.8 allyl groups for each sucrose molecule. Also suitable for use herein are hydrophobically-modified crosslinked polymers of acrylic acid having amphipathic properties available under the Trade Name Carbopol 1382, Carbopol 1342 and Pemulen TR-1. A combination of the polyalkenyl polyether cross-linked acrylic acid polymer and the hydrophobically modified crosslinked acrylic acid polymer is also suitable for use herein.

Other suitable gelling agents suitable for use herein are oleogels such as trihydroxystearin and aluminum magnesium hydroxy stearate.

In some embodiments, the gelling agent is present in the cosmetic/cleansing formulation in an amount from about 0.01% to about 10% of the total weight of the formulation. In some embodiments, the formulation comprises a hydrophilic gelling agent in an amount between about 0.02% to about 2%. In other embodiments, the amount of the gelling agent is from about 0.02% to about 0.5%.

The cosmetic/cleansing formulation may also comprise a thickener, such as crosslinked maleic anhydride-alkyl methylvinylethers, and copolymers, commercially available as Stabilizes QM (International Specialty Products (ISP)), Carbomer, natural gums, highly crosslinked polymethacrylate copolymer, such as Microspongess 5647, which take the form of generally spherical particles of crosslinked hydrophobic polymer having a pore size of from about 0.01 to about 0.05 μm and a surface area of 200-300 m²/g.

Neutralizing agents suitable for use in a cosmetic/cleansing formulation include neutralizing acidic group containing hydrophilic gelling agents, as listed herein, sodium hydroxide, potassium hydroxide, ammonium hydroxide, monoethanolamine, diethanolamine and triethanolamine and aminomethyl propanol.

In some embodiments, the cosmetic/cleansing formulation comprises one or more ultraviolet absorbing agents. Ultraviolet absorbing agents, often described as sun screening agents, may be present in a concentration between about 1% and about 25% by weight, based on the total weight of composition. According to some embodiments, the UV absorbing agent constitutes between about 2% and 15% by weight. According to other embodiments, the UV absorbing agent constitutes between about 4% and about 10% by weight. Non-limiting examples of ultraviolet absorbing agents include benzophenone-3, benzophenone-4, octyl dimethyl PABA (Padimate 0), octyl methoxy cinnamate, octyl salicylate, octocrylene, p-methylbenzylidene camphor, butyl methoxy dibenzoyl methane (Parsol 1789), titanium dioxide, zinc oxide and mixtures thereof.

In a further aspect, the disclosure provides an antimicrobial formulation comprising the compositions disclosed herein.

The antimicrobial formulation is effective in reducing or eliminating a microorganism population or a biofilm of such microorganisms. As demonstrated herein, the formulations provide antimicrobial activity against a wide spectrum of microorganisms and specifically against a broad spectrum of bacteria. The term microorganism relates herein to a single cell (unicellular), cell clusters, or no cell (acellular) organism such as bacteria, fungi, yeast, mold, archaea, protists, viruses and algae.

In some embodiments, the microorganism is a bacteria, being selected, in some embodiments from Acinetobacter baumannii, Anaerococcus sp, Anaerococcus prevotii, Bordetella pertussis, Borrelia burgdorferi, Brucella abortus, Brucella canis, Brucella melitensis, Brucella suis, Burkholderia cepacia, Campylobacter jejuni, Chlamydia pneumonia, Chlamydia psittaci, Chlamydia trachomatis, Clostridium botulinum, Clostridium difficile, Clostridium perfringens, Clostridium tetani, Corynebacterium diphtheria, Cutibacterium acnes, Enterobacter cloacae, Enterobacter cloacae, Enterococcus faecalis, Enterococcus faecium, Escherichia coli (E. coli), Enterotoxigenic Escherichia coli (ETEC), Enteropathogenic E. coli, Francisella tularensis, Gardnerella vaginalis, Haemophilus influenza, Halomonas elongate, Helicobacter pylori, Klebsiella oxytoca, Lactobacillus acidophilus, Legionella pneumophila, Leptospira interrogans, Listeria monocytogenes, Mycobacterium leprae, Mycobacterium tuberculosis, Mycoplasma pneumonia, Neisseria gonorrhoeae, Neisseria meningitidis, Pseudomonas aeruginosa, Rickettsia rickettsii, Salmonella typhi, Salmonella typhimurium, Shigella sonnei, Staphylococcus capitis, Staphylococcus epidermidis, Staphylococcus saprophyticus, Streptococcus agalactiae, Streptococcus mutans Streptococcus pneumonia, Streptococcus pyogenes, Treponema pallidum, Vibrio cholera, Vibrio harveyi and Yersinia pestis.

In some embodiments, the microorganism is a fungus, selected in some embodiments from Absidia corymbifera, Ajellomyces capsulatus, Ajellomyces dermatitidis, Arthroderma benhamiae, Arthroderma fulvum, Arthroderma gypseum, Arthroderma incurvatum, Arthroderma otae, Arthroderma vanbreuseghemii, Aspergillus flavus, Aspergillus fumigates, Aspergillus niger (Aspergillus brasiliensis), Aspergillus terreus, Blastomyces dermatitidis, Candida albicans, Candida albicans var. stellatoidea, Candida dublinensis, Candida glabrata, Candida guilliermondii, Candida krusei, Candida parapsilosis, Candida pelliculosa, Candida tropicalis, Cladophialophora carrionii, Coccidioides immitis, Cryptococcus neoformans, Cunninghamella sp., Epidermophyton floccosum, Exophiala dermatitidis, Filobasidiella neoformans, Fonsecaea pedrosoi, Geotrichum candidum, Histoplasma capsulatum, Hortaea werneckii, Issatschenkia orientalis, Madurella grisae, Malassezia furfur, Malassezia furfur complex, Malassezia globosa, Malassezia obtuse, Malassezia pachydermatis, Malassezia restricta, Malassezia slooffiae, Malassezia sympodialis, Microsporum canis, Microsporum fulvum, Microsporum gypseum, Microsporum gypseum complex, Microsporum gypseum, Mucor circinelloides, Nectria haematococca, Paecilomyces variotii, Paracoccidioides brasiliensis, Penicillium marneffei, Phialophora verrucosa, Pichia anomala, Pichia guilliermondii, Pneumocystis jirovecii, Pseudallescheria boydii, Rhizopus oryzae, Rodotorula rubra, Saccharomyces cerevisiae, Scedosporium apiospermum, Schizophyllum commune, Sporothrix schenckii, Stachybotrys chartarum, Trichophyton mentagrophytes, Trichophyton mentagrophytes complex, Trichophyton mentagrophytes, Trichophyton mentagrophytes, Trichophyton rubrum, Trichophyton tonsurans, Trichophyton verrucosum, Trichophyton violaceum, Trichosporon asahii, Trichosporon cutaneum, Trichosporon cutaneum complex, Trichosporon inkin, Trichosporon mucoides and Wickerhamomyces anomalus.

In some embodiments, the microorganism is yeast, being selected, in some embodiments, from Candida albicans, Candida albicans var. stellatoidea, Candida dublinensis, Candida glabrata, Candida guilliermondii, Candida krusei, Candida parapsilosis, Candida pelliculosa, Candida tropicalis, Cryptococcus neoformans, Filobasidiella neoformans, Geotrichum candidum, Issatschenkia orientalis, Malassezia furfur, Malassezia pachydermatis, Pichia anomala, Pichia guilliermondii, Pityrosporum ovale, Pneumocystis jirovecii, Rodotorula rubra, Trichosporon asahii, Trichosporon cutaneum, Trichosporon inkin and Trichosporon mucoides.

In some embodiments, the microorganism is mold, being selected, in some embodiments, from Absidia corymbifera, Arthroderma benhamiae, Arthroderma fulvum, Arthroderma gypseum, Arthroderma incurvatum, Arthroderma otae, Arthroderma vanbreuseghemii, Aspergillus flavus, Aspergillus fumigates, Aspergillus niger, Cladophialophora carrionii, Coccidioides immitis, Epidermophyton floccosum, Exophiala dermatitidis, Fonsecaea pedrosoi, Hortaea werneckii, Madurella grisae, Microsporum canis, Microsporum fulvum, Microsporum gypseum, Microsporum gypseum, Microsporum gypseum, Mucor circinelloides, Nectria haematococca, Paecilomyces variotii, Paracoccidioides brasiliensis, Penicillium marneffei, Pseudallescheria boydii, Rhizopus oryzae, Scedosporium apiospermum, Schizophyllum commune, Sporothrix schenckii, Stachybotrys chartarum, Trichophyton mentagrophytes complex, Trichophyton mentagrophytes, Trichophyton mentagrophytes, Trichophyton rubrum, Trichophyton tonsurans, Trichophyton verrucosum and Trichophyton violaceum.

According to some embodiments of this disclosure, the antimicrobial formulations disclosed herein are effective against bacteria such as Escherichia coli (E. Coli), Salmonella, Staphylococcus, Saccharomyces, Staphylococcus aureus, Pseudomonas aeruginosa, Candida albicans, A. niger, L. acidophilus, Aspergillus terreus, Klebsiella oxytoca, Penicillium marneffei, Acinetobacter baumannii, Pityrosporum ovale, Staphylococcus capitis, Wickerhamomyces anomalus, Staphylococcus saprophyticus, Anaerococcus prevotii, Staphylococcus epidermidis, Streptococcus mutans, Cutibacterium acnes, Halomonas elongate, and/or Gardnerella vaginalis.

According to some embodiments of this disclosure, the antimicrobial formulations disclosed herein are effective against bacteria such as Escherichia coli (E. Coli), Salmonella, Staphylococcus, Saccharomyces, Staphylococcus aureus, Pseudomonas aeruginosa, Candida albicans, A. niger.

By some embodiments, the compositions of this disclosure exhibit delayed antimicrobial activity. In other words, the compositions are designed to eliminate undesired microorganisms in the long term (for example, after a few days from exposure), however have no effect, or have significantly reduced effect, when exposing the microorganisms to them for short periods of time. The compositions do not have significant antimicrobial effect in short-term exposure, making the compositions friendly to the skin microbiota for a period of time suitable for the application of the composition onto the skin, however showing an antimicrobial effect after longer exposure, for example in storing conditions of the cosmetic product into which the compositions are combined (e.g. creams, lotions, gels, etc.). Such delayed antimicrobial effect enables providing a composition which can be used to preserve cosmetic formulations for a prolonged period of time against the growth of undesired microbiological contaminants (such as Escherichia coli (E. Coli), Staphylococcus aureus, Pseudomonas aeruginosa, Candida albicans, A. niger) when the microorganisms are continuously exposed to the composition, while maintaining the desired and beneficial microbiota of the skin (e.g. S. capitis, S. epidermis or S. hominis) when the cosmetic formulation is applied thereto.

Therefore, by another aspect of this disclosure, there is provided a composition having a delayed antimicrobial activity, the composition comprising at least one saponin extract, an extract from at least one plant species selected from the genus Styrax (a Styrax extract), the genus Myroxylon (a Miroxylon extract), the genus Myrocarpus (a Myrocarpus extract) and mixtures thereof, and at least one additional plant extract from an additional plant source, the saponin extract being from a saponin source different from said plant species of the genera Styrax, Myroxylon, or Myrocarpus, and from said additional plant source.

By some embodiments, the composition shows antimicrobial activity observable from at least 5 hours of continuous exposure of the microorganisms to the composition. By other embodiments, the composition shows antimicrobial activity starting from at least 8 hours, 12 hours, 24 hours, 2-days, 3-days, 4-days or even 5-days of continuous exposure of the microorganisms to the composition.

By some embodiments, the additional plant extract is selected from Cinnamomum extract, Paeonia extract, Salvia extract, Magnolia extract, Commiphora extract, Boswellia extract, Dipterocarpus extract, Copaiba extract, Malpighia extract, Thymus extract, Origanum extract, Cymbopogon extract, Anethum extract, Syzygium extract, Nigella extract, Pimenta extract, and maltol extracted from a plant source, and mixtures thereof.

In a further aspect, the disclosure provides a therapeutic formulation (pharmaceutical composition) comprising the compositions of the present disclosure as described herein.

The pharmaceutical formulation of the disclosure may be effective in the treatment and/or prevention of a variety of diseases and disorders. As demonstrated hereinbelow, the formulations provide antimicrobial activity against a wide spectrum of microorganisms, as defined herein. In some embodiments, the disease or disorder to be treated is associated with bacterial infection, fungal infection or viral infection.

Non-limiting examples of disease or disorder associated with a bacterial infection include lyme disease, brucellosis, acute enteritis, psittacosis, nongonococcal urethritis (NGU), trachoma, inclusion conjunctivitis of the newborn (ICN), lymphogranuloma venereum (LGV), botulism, pseudomembranous colitis, gas gangrene, acute food poisoning, anaerobic cellulitis, tetanus, diphtheria, nosocomial infections, urinary tract infections (UTI), diarrhea, meningitis, meningitis in infants, hemorrhagic colitis, hemolytic-uremic syndrome, tularemia, upper respiratory tract infections, pneumonia, mycoplasma pneumonia, secondary pneumonia, bronchitis, peptic ulcer, legionnaire's disease, gastric B-cell lymphoma, pontiac fever, leptospirosis, listeriosis, leprosy (Hansens disease), tuberculosis, gonorrhea, ophthalmia neonatorum, meningococcal disease, Waterhouse-Friderichsen, localized infection (of eye, ear, skin, urinary, respiratory), dental caries, gastrointestinal tract infection, central nervous system infection, systemic infection with bacteremia, bone and joint infections, endocarditis, typhoid fever type salmonellosis, dysentery, colitis, salmonellosis with gastroenteritis and enterocolitis, bacillary dysentery/shigellosis, Streptococcal pharyngitis, Scarlet fever, rheumatic fever, impetigo and erysipelas, puerperal fever, necrotizing fasciitis, syphilis, congenital syphilis and cholera.

In some embodiments, the bacterial disease or disorder is associated with Staphylococcus or Escherichia coli (E. coli) or salmonella infections; the disease or disorder being selected from:
Staphylococcus: coagulase-positive staphylococcal infections such as Localized skin infections, diffuse skin infection (impetigo), deep and localized infections, acute infective endocarditis, septicemia, necrotizing pneumonia, toxinoses, toxic shock syndrome, staphylococcal food poisoning, infections of implanted prostheses e.g., heart valves and catheters and cystitis in women;
E. coli: urinary tract infections (UTI), diarrhea, meningitis in infants, traveler's diarrhea, hemorrhagic colitis and hemolytic-uremic syndrome;
Salmonella: typhoid fever type salmonellosis, dysentery, colitis, salmonellosis, e.g., with gastroenteritis and enterocolitis.

In some embodiments, the pharmaceutical composition is used in the treatment or prevention of a disease or disorder associated with a fungal infection. In some embodiments, the pathogen is yeast. In some other embodiments, the pathogen is mold.

The pharmaceutical composition may be adapted for administration by a variety of routes including topical, oral, rectal, vaginal, transdermal, subcutaneous, intravenous, intramuscular, eye drops and intranasal. Such pharmaceutical composition is prepared in a manner well known in the pharmaceutical art. In making the pharmaceutical composition, the aforementioned components are usually mixed with an excipient, diluted by an excipient or enclosed within such a carrier which can be manipulated to the desired form. Based on the particular mode of administration, the pharmaceutical composition may be formulated into tablets, pills, capsules, sachets, granules, powders, chewing gum, suspensions, emulsions and solutions.

The pharmaceutically acceptable carriers, for example, vehicles, adjuvants, excipients, or diluents, are well-known to those who are skilled in the art and are readily available to the public. It is preferred that the pharmaceutically acceptable carrier be one which is chemically inert to the active formulation and each of its components and one which has no detrimental side effects or toxicity under the conditions of use.

The choice of carrier will be determined in part by the particular formulation, as well as by the particular method used to administer the composition. Accordingly, there is a wide variety of suitable formulations of the pharmaceutical composition.

Formulations suitable for oral administration can consist of (a) liquid solutions, such as an effective amount of the compound, or composition comprising same, dissolved in diluents, such as water, saline, or juice (e.g. orange juice); (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active formulation in an inert base, such as gelatin and glycerin, or sucrose and acacia, emulsions, gels, and the like containing, in addition to the active formulation, such carriers as are known in the art.

The formulations of the present disclosure, alone or in combination with other suitable components, e.g., active or non-active additives/ingredients can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. The formulation can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical additives.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-p-aminopriopionates, and 2-alkyl-imidazoline quaternary ammonium salts, and (3) mixtures thereof.

In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The active formulation is effective over a wide dosage range and may generally be administered in a pharmaceutically effective amount. It should be understood, however, that the amount of the formulation or each component thereof to be administered, will be determined by a physician, in the light of the relevant circumstances, including the condition to be treated, the chosen route of administration, the actual formulation, the age, weight, and response of the individual patient, the severity of the patient's symptoms, and the like.

In another aspect of this disclosure, there is provided the use of a formulation of this disclosure as herein defined, for the preparation of a pharmaceutical composition for treating or preventing a disease or disorder in a mammal (human or non-human).

In another aspect of the disclosure, there is provided the use of a topical formulation of the disclosure as herein defined, for the preparation of a pharmaceutical composition for treating or preventing a disease or disorder in a mammal (human or non-human).

In some embodiments, the disease or disorder is associated with a bacteria, virus, fungus, yeast or mold.

As used herein, the term treatment or any lingual variation thereof, refers to the administering of a therapeutic amount of the composition which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above. The effective amount for purposes disclosed herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half life in the body, on undesired side effects, if any, on factors such as age and gender, etc.

In yet another aspect, the disclosure provides a preservative formulation comprising the compositions of the disclosure as described herein.

The preservative formulation of this disclosure may be used to reduce, inhibit or completely eliminate pathogen population in a variety of consumer products, such as personal care products, industrial products, food products, therapeutics, and others. As demonstrated herein, the formulation may be used to replace currently available chemicals which are used as preservatives, some of which known as toxic to humans and animals, or at reduce their concentration in such products for human or animal use. The preservative formulation may be added to any such product, such as cosmetics and toiletries in aqueous or hydroalcoholic solution, oil-in-water or water-in-oil emulsion, aqueous or anhydrous gels, cream, ointment, lotion, gel, solution and suspension; therapeutics and over-the-counter pharmaceutical products, water-based paints, cutting oils, latex solutions, food products such as beverages, frozen foods, candy and canned products.

In some embodiments, the formulation of this disclosure is an antimicrobial preservative, attesting to the ability of the formulations to suppress microbial growth, reduce microbial infestation, treat products or surfaces to improve product resistance to microbial infestation, reduce biofilm, remove biofilm, prevent conversion of bacteria to biofilm, prevent or inhibit microbial infection, prevent spoilage, retard or minimize or prevent quorum sensing, and retard microbial reproduction. Typically, the preservative formulation according to the disclosure comprises the saponin and plant extracts at a concentration which suffices to prevent spoilage or growth of microorganisms, thereby extending the shelf- or useful-life of the product.

The formulation of this disclosure may also be employed as a disinfectant or bacteriocide agent. The formulations of the disclosure may be applied onto a surface to be disinfected, including human or animal skin, by various means including by washing, spraying, wiping, etc.

As used herein, the term about is meant to encompass deviation of ±10% from the specifically mentioned value of a parameter, such as temperature, pressure, concentration, etc.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

For the avoidance of doubt, all concentrations of ingredients in this disclosure are provided as weight-per-weight (i.e. weight percent or wt %), unless otherwise explicitly and/or specifically noted

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1D are pictures agar plates containing body lotion samples taken after 3-4 hours of incubation with a mixture of representative microorganisms from skin microbiota. The upper panel of each agar plate was plated with mixture of representative microorganisms to provide a control to each of the experiments. The lower panel of each agar plate represent cream (preserved or unpreserved) which was inoculated with the same amount of microorganisms mixture: FIG. 1A bottom part of the plate is an unpreserved cream; FIG. 1B bottom part of the plate shows use of a commercial synthetic preservative; FIG. 1C bottom part of the plate shows use of Composition 5A according to Example 5; and FIG. 1D bottom part of the plate shows use of Composition 5B according to Example 5.

### DETAILED DESCRIPTION OF EMBODIMENTS

### Example 1: Composition of Saponin Extract, Styrax Extract and Maltol in Cream Formulation

A Preservatives Effectiveness Test (PET) was carried out for various combinations of saponin extract obtained from Sapindus mukorossi, Styrax extract (Sumatra benzoin) and maltol (obtained from fermented wheat) for evaluating the synergistic antimicrobial effect therebetween. The antimicrobial efficiency was tested in accordance with current ISO 11930 requirements. The test consists of challenging a reference cream lotion with a prescribed inoculum of suitable microorganisms. The tested microorganisms used for the PET are detailed in Table 1.

TABLE 1
Tested microorganisms

| | | | Culture | PET |
|---|---|---|---|---|
| | | Culture | Growth | Growth |
| Microorganism | ATCC | Media | Conditions | Conditions |
| Staphylococcus | 6538 | TSA | 18-24 hr, | 3-5 days, |
| aureus (SA) | | | 32.5 ± 2.50 C. | 32.5 ± 2.50 C. |
| Escherichia | 8739 | | | |
| coli (EC) | | | | |
| Pseudomonas | 9027 | | | |
| aeruginosa (PA) | | | | |
| Candida | 10231 | SDA | 48-72 hr, | 5-7 days, |
| albicans (CA) | | | 22.5 ± 2.50 C. | 22.5 ± 2.50 C. |
| Aspergillus | 16404 | | 7 days, | |
| brasiliensis (AB) | | | 22.5 ± 2.50 C. | |

Each sample was inoculated at time “0” and mixed with one of the microbial suspensions using a ratio-equivalent to 1% inoculum to product (200 μl inoculum into 20 g sample). The concentrations of test microorganisms added to the cream were such that the concentration in the cream immediately after inoculation was between 1×10⁵-1×10⁶ CFU/g for bacteria and 1×10⁴-1×10⁵ CFU/g for molds.

Samples were stored at 20-25° C. The samples were sampled in intervals of 7, 14 and 28 days. At each time interval, 0.5 g of the sample was diluted 1:10 in TAT (neutralizer buffer). 100 μl of each diluted sample was seeded using the Pour Plate method on TSA plates for bacteria and on SDA plates for the fungi. Each plating was done in duplicates. For A. brasiliensis at Day 7 only, a second dilution of 1:10 was prepared in TAT before seeding (for a total of 1:100 dilution).

The number of viable microorganisms/g in the mixture at each test interval was determined by the Pour Plate Count Method. The TSA plates were incubated at 30-35° C. for 3-5 days unless a reliable count was obtained in a shorter time. The SDA plates were incubated at 20-25° C. for 5-7 days or less if reliable count was obtained prompter.

The counts obtained in all countable dilutions were used to calculate the mean value of CFU, per gram product/challenge microorganism/time interval. Using the calculated concentration of microorganisms in the initial inoculum per gram present as a baseline, the change in log 10 values of the concentration of the CFU/g was calculated for each time interval and the changes were expressed in terms of log reduction.

$$Log Reduction = \frac{Log ( Initial count of each microorganism )}{Log ( countable microorganisms at each time interval )}$$

Log reduction results are provided in Table 2. In the Tables below, AB, CA, PA, EC and SA indicate A. brasilliensis, C. albicans, P. aeruginosa, E. coli and S. aureus, respectively.

TABLE 2
Summary of results for saponin extract (SN), Styrax extract (BZ) and
maltol (ML) combinations, log reduction from initial inoculation

| Formula (wt %) | | | | 7 Days | | | | 14 Days | | | | 28 Days | | | |
|---|---|---|---|---|---|---|---|---|---|---|---|---|---|---|---|
| # | SN | BZ | ML | AB | CA | SA | PA | AB | CA | SA | PA | AB | CA | SA | PA |
| 1 | 1.5 | 0.5 | 2 | — | — | 5.39 | 5.37 | 5.41 | 5.69 | 5.39 | 5.37 | 5.41 | 5.69 | 5.39 | 5.39 |
| 2 | 1.5 | 0.5 | — | — | — | 5.39 | 0 | 1.54 | 5.69 | 5.39 | 0 | 2.8 | 0 | 5.39 | 0 |
| 3 | 1.5 | 0.5 | 0.2 | — | — | 5.39 | 5.37 | 1.64 | 5.69 | 5.39 | 5.37 | 3.01 | 5.69 | 5.39 | 5.39 |
| 4 | — | — | 2 | — | — | 5.39 | 5.37 | 5.41 | 5.69 | 5.39 | 5.37 | 5.41 | 5.69 | 5.39 | 5.39 |
| 5 | 0.15 | 0.05 | 2 | — | — | 5.39 | 5.37 | 5.41 | 5.69 | 5.39 | 5.37 | 5.41 | 5.69 | 5.39 | 5.39 |
| 6 | 0.75 | 0.25 | 1 | — | — | 5.39 | 5.37 | 5.41 | 5.69 | 5.39 | 5.37 | 5.41 | 5.69 | 5.39 | 5.39 |
| 7 | 0.75 | 0.25 | — | — | — | 5.39 | 0 | 0 | 5.69 | 5.39 | 0 | 0 | 0 | 5.39 | 0 |
| 8 | 0.75 | 0.25 | 0.2 | — | — | 5.39 | 2.35 | 1.46 | 5.69 | 5.39 | 5.37 | 0 | 5.69 | 5.39 | 5.39 |
| 9 | — | — | 1 | — | — | 5.39 | 5.37 | 5.41 | 5.69 | 5.39 | 5.37 | 5.41 | 5.69 | 5.39 | 5.39 |
| 10 | 0.15 | 0.05 | 1 | — | — | 5.39 | 5.37 | 5.41 | 5.69 | 5.39 | 5.37 | 5.41 | 5.69 | 3.69 | 5.39 |
| 11 | — | — | 0.2 | — | — | 0 | 0 | 1.16 | 5.69 | 1.59 | 0 | 0 | 5.69 | 5.39 | 0 |
| 12 | 0.15 | 0.05 | 0.2 | — | — | 5.39 | 0 | 1.22 | 5.69 | 5.39 | 0 | 0 | 5.69 | 5.39 | 0 |
| 13 | 0.15 | 0.05 | — | — | — | 5.39 | 0 | 0 | 0 | 5.39 | 0 | 0 | 0 | 5.39 | 0 |
| 14 | 0.15 | 0.05 | 0.04 | — | — | 5.39 | 0 | 1.85 | 5.69 | 5.39 | 0 | 1.86 | — | 5.39 | 0 |
| 15 | — | — | 0.04 | — | — | 0 | 0 | 0 | 0 | 0 | 0 | 0 | 0 | 3.69 | 0 |
| 16 | 0.03 | 0.01 | 0.2 | — | — | 0 | 0 | 0 | 5.69 | 2.41 | 0 | 0 | 5.69 | 5.39 | 0 |
| 17 | 0.03 | 0.01 | — | — | — | 0 | 0 | 0 | 0 | 1.96 | 0 | 0 | 0 | 0 | 0 |
| 18 | 0.03 | 0.01 | 0.04 | — | — | 0 | 0 | 1.04 | 0 | 3.39 | 0 | 0 | 0 | 5.39 | 0 |
| 19 | 0.15 | 0.05 | 0.02 | — | — | 5.39 | 0 | 1.82 | 0 | 5.39 | 0 | 1.7 | 0 | 3.69 | 0 |
| 20 | — | — | 0.02 | — | — | 0 | 0 | 0 | 0 | 0 | 0 | 0 | 0 | 3.69 | 0 |
| 21 | 0.015 | 0.005 | 0.2 | — | — | 0 | 0 | 0 | 5.69 | 1.54 | 0 | 0 | 5.69 | 5.39 | 0 |
| 22 | 0.015 | 0.005 | — | — | — | 0 | 0 | 1.26 | 0 | 1.37 | 0 | 0 | 0 | 3.69 | 0 |
| 23 | 0.015 | 0.005 | 0.02 | — | — | 0 | 0 | 1.02 | 0 | 1.93 | 0 | 0 | 0 | 5.39 | 0 |

| | | | | | | | | | | | | | |
|---|---|---|---|---|---|---|---|---|---|---|---|---|---|
| 24 | Synthetic Preservative 1% | — | — | 5.39 | 5.37 | 5.41 | 5.69 | 5.39 | 5.37 | 5.41 | 5.69 | 5.39 | 5.39 |
| 25 | cream 100% | 0.0 | 0.0 | 0.0 | 0.0 | 0.0 | 0.0 | 0.0 | 0.0 | 0.0 | 0.0 | 0.0 | 0.0 |

For P. aeruginosa, a synergistic effect of the SN/BZ/ML combination was seen at 0.2 wt % maltol combined with 0.75 wt % saponin extract and 0.25 wt % Styrax extract. For A. brasilliensis, a synergistic effect of the combination was seen at 0.04 wt % maltol combined with 0.15 wt % saponin extract and 0.05 wt % Styrax extract. For C. albicans, a synergistic effect was seen at 0.04 wt % maltol combined with 0.15 wt % saponin extract and 0.05 wt % Styrax extract.

### Example 2: Composition of Saponin Extract, Styrax Extract, Maltol and Citric Acid in Cream Formulation

A Preservatives Effectiveness Test (PET) was carried out for various combinations of saponin extract from Sapindus mukorossi, Styrax extract (Sumatra benzoin), maltol (obtained from fermented wheat) and citric acid as an additive. The test was carried out similarly to Example 1. Log reduction results are provided in Table 3.

TABLE 3
Summary of results for saponin extract (SN), Styrax extract (BZ), maltol (ML)
and citric acid (cit) combinations, log reduction from initial inoculation

| Formula (wt %) | | | | | 7 Days | | | | 14 Days | | | | 28 Days | | | |
|---|---|---|---|---|---|---|---|---|---|---|---|---|---|---|---|---|
| # | SN | BZ | ML | CIT | AB | CA | SA | PA | AB | CA | SA | PA | AB | CA | SA | PA |
| 1 | 3.75 | 1.25 | 2 | 0.15 | 5.5 | 5.1 | 5.8 | 6.7 | 5.5 | 5.1 | 5.8 | 6.7 | 5.5 | 5.1 | 5.8 | 6.7 |
| 2 | — | — | — | 0.15 | 1.4 | 0 | 0 | 0 | 1.7 | 0 | 0 | 0 | 1.9 | 0 | — | 0 |
| 3 | 0.375 | 0.125 | 0.2 | 0.15 | 0 | 1.4 | 5.8 | 0 | 1.4 | 5.1 | 5.8 | 0 | 1.9 | 5.1 | 5.8 | 6.7 |
| 4 | 3.75 | 1.25 | 2 | — | 5.5 | 5.1 | 5.8 | 6.7 | 5.5 | 5.1 | 5.8 | 2.7 | 5.5 | 5.1 | 5.8 | 6.7 |
| 5 | 3.75 | 1.25 | 2 | 0.015 | 5.5 | 5.1 | 5.8 | 6.7 | 5.5 | 5.1 | 5.8 | 4.1 | 5.5 | 5.1 | 5.8 | 6.7 |
| 6 | 1.875 | 0.625 | 1 | 0.075 | 5.5 | 5.1 | 5.8 | 6.7 | 5.5 | 5.1 | 5.8 | 6.7 | 5.5 | 5.1 | 5.8 | 6.7 |
| 7 | — | — | — | 0.075 | 1 | 0 | 0 | 0 | 1.6 | 0 | 0 | 0 | 1.5 | 0 | — | 0 |
| 8 | 0.375 | 0.125 | 0.2 | 0.075 | 0 | 2.4 | 5.8 | 0 | 0 | 5.1 | 5.8 | 5.2 | 0 | 5.1 | 5.8 | 6.7 |
| 9 | 1.875 | 0.625 | 1 | — | 5.5 | 5.1 | 5.8 | 6.7 | 5.5 | 5.1 | 5.8 | 6.7 | 5.5 | 5.1 | 5.8 | 6.7 |
| 10 | 1.875 | 0.625 | 1 | 0.015 | 5.5 | 5.1 | 5.8 | 6.7 | 5.5 | 5.1 | 5.8 | 6.7 | 5.5 | 5.1 | 5.8 | 6.7 |
| 11 | 0.375 | 0.125 | 0.2 | — | 1 | 1.9 | 5.8 | 6.7 | 2.1 | 5.1 | 5.8 | 6.7 | 3.1 | 5.1 | 5.8 | 6.7 |
| 12 | 0.375 | 0.125 | 0.2 | 0.015 | 0.7 | 1.7 | 5.8 | 6.7 | 1.6 | 5.1 | 5.8 | 6.7 | 2.2 | 5.1 | 5.8 | 6.7 |
| 13 | — | — | — | 0.015 | 0.8 | 0 | 0 | 0 | 1.5 | 0 | 0 | 0 | 1.4 | 0 | — | 0 |
| 14 | 0.075 | 0.025 | 0.04 | 0.015 | 2.8 | 0 | 5.8 | 0 | 2.5 | 0 | 5.8 | 0 | 2.4 | 0 | 5.8 | 0 |
| 15 | 0.075 | 0.025 | 0.04 | — | 2.3 | 0 | 5.8 | 0 | 2.3 | 0 | 5.8 | 0 | 1.9 | 0 | 5.8 | 0 |
| 16 | 0.375 | 0.125 | 0.2 | 0.003 | 1.2 | 1.9 | 5.8 | 6.7 | 2.4 | 5.1 | 5.8 | 6.7 | 5.5 | 51 | 5.8 | 6.7 |
| 17 | — | — | — | 0.003 | 0.7 | 0 | 0 | 0 | 0 | 0 | 0 | 0 | 0 | 0 | 0 | 0 |
| 18 | 0.075 | 0.025 | 0.04 | 0.003 | 2.1 | 1.2 | 5.8 | 0 | 2.4 | 1.2 | 5.8 | 0 | 2.2 | 0 | 5.8 | 0 |
| 19 | 0.0375 | 0.0125 | 0.02 | 0.015 | 1.2 | 0 | 5.8 | 0 | 1.4 | 0 | 5.8 | 0 | 0 | 0 | 5.8 | 0 |
| 20 | 0.0375 | 0.0125 | 0.02 | — | 1.5 | 0 | 0 | 0 | 2.1 | 0 | 5.8 | 0 | 1.9 | 0 | 5.8 | 0 |
| 21 | 0.375 | 0.125 | 0.2 | 0.0015 | 1 | 1.7 | 5.8 | 6.7 | 2.3 | 5.1 | 5.8 | 3.4 | 2.9 | 5.1 | 5.8 | 6.7 |
| 22 | — | — | — | 0.0015 | 0.5 | 0 | 0 | 0 | 0 | 0 | 0 | 0 | 0 | 0 | 0 | 0 |
| 23 | 0.0375 | 0.0125 | 0.02 | 0.0015 | 1.4 | 0 | 5.8 | 0 | 1.7 | 0 | 5.8 | 0 | 1.6 | 0 | 5.8 | 0 |

| | | | | | | | | | | | | | |
|---|---|---|---|---|---|---|---|---|---|---|---|---|---|
| 24 | Synthetic Preservative 1% | 5.5 | 5.1 | 5.8 | 6.7 | 5.5 | 5.1 | 5.8 | 6.7 | 5.5 | 5.1 | 5.8 | 6.7 |
| 25 | cream 100% | 0.0 | 0.0 | 0.0 | 0.0 | 0.0 | 0.0 | 0.0 | 0.0 | 0.0 | 0.0 | 5.8 | 0.0 |

For C. albicans, a synergistic effect of the combination was seen at 0.04 wt % maltol combined with 0.075 wt % saponin extract, 0.025 wt % Styrax extract and 0.003 wt % citric acid. For S. aureus, a synergistic effect of the combination was seen at 0.02 wt % maltol combined with 0.0375 wt % saponin extract, 0.0125 wt % Styrax extract and 0.0015 wt % citric acid.

### Example 3: Composition of Saponin and Styrax Extracts, with Maltol as an Additional Plant Extract in Agar Dilution Test, with and without Citric Acid

An agar dilution test was carried out for various combinations of saponin extract (Sapindus mukorossi) and Styrax extract (Sumatra benzoin), with maltol extracted from wheat as an additional plant extract, for evaluating the synergistic antimicrobial effect therebetween.

The agar dilution test was carried out as follows: TSA (tryptic soy agar) or SDA (Sabouraud dextrose agar) media were melted at 99±2° C. and kept warm until seeding (43±2° C.). Into a quota of the growth medium, the maltol and extract were added and stirred. 5 ml of the mixture was poured into each test plate and left to cool and solidify. The plates were then inoculated by defined amounts of test microorganisms (E. coli, S. aureus and P. aeruginosa used on TSA plates, C. albicans and A. brasilliensis used on the SDA plates). The plates were incubated at 25-35° C. for 24-48 hours and then assessed for microorganisms' growth. The agar dilution test provides test results which are independent of the formulation (e.g. cosmetic formulation) into which the composition is to be incorporated, hence can be used as a good basis for comparing activity of the compositions.

The test results for the combinations of saponin and Styrax extracts with maltol are detailed in Table 4, while the results for the compositions of saponin and Styrax extracts with maltol and citric acid are shown in Table 5. (+) indicates growth, (+/−) indicates partial growth, while (−) indicates no growth. Positive control was obtained by utilizing a commercial preservative.

TABLE 4
Agar dilution test results for compositions of saponin (SN)
and Styrax (BZ) extracts with maltol (ML) (amounts in wt %)

| | Test 1 | | | | | Teat 2 (duplicate) | | | | |
|---|---|---|---|---|---|---|---|---|---|---|
| | AB | CA | PA | EC | SA | AB | CA | PA | EC | SA |
| ML 0.02% | + | + | + | + | + | + | + | + | + | + |
| ML 0.04% | + | +/− | + | + | +/− | + | +/− | + | + | +/− |
| ML 0.05% | + | + | +/− | + | +/− | + | + | +/− | + | +/− |
| ML 0.08% | +/− | − | − | +/− | + | +/− | − | − | +/− | + |
| ML 0.1% | +/− | − | − | + | − | +/− | − | − | + | − |
| ML 0.2% | − | − | − | − | − | − | − | − | − | − |
| SN 0.075% + BZ 0.025% | +/− | +/− | + | + | +/− | +/− | +/− | + | + | +/− |
| SN 0.15% + BZ 0.05% | +/− | +/− | + | + | +/− | +/− | +/− | + | + | +/− |
| SN 0.075% + BZ 0.025% + ML 0.04% | +/− | − | + | + | − | +/− | − | + | + | − |
| SN 0.15% + BZ 0.05% + ML 0.04% | +/− | − | + | + | − | +/− | − | + | + | − |
| SN 0.075% + BZ 0.025% + ML 0.05% | +/− | +/− | +/− | + | − | +/− | +/− | +/− | + | − |
| SN 0.15% + BZ 0.05% + ML 0.05% | +/− | − | +/− | + | − | +/− | − | +/− | + | − |
| SN 0.15% + BZ 0.05% + ML 0.075% | +/− | − | +/− | + | +/− | +/− | − | +/− | + | +/− |
| SN 0.075% + BZ 0.025% + ML 0.08% | +/− | − | +/− | + | + | +/− | − | +/− | + | + |
| SN 0.15% + BZ 0.05% + ML 0.08% | +/− | − | +/− | + | − | +/− | − | +/− | + | + |
| SN 0.075% + BZ 0.025% + ML 0.1% | − | − | − | + | − | − | − | − | + | − |
| SN 0.15% + BZ 0.05% + ML 0.15% | − | − | − | +/− | − | − | − | − | +/− | − |
| SN 0.075% + BZ 0.025% + ML 0.2% | − | − | − | − | − | − | − | − | − | − |
| SN 0.15% + BZ 0.05% + ML 0.2% | − | − | − | − | − | − | − | − | − | − |
| Positive control | − | − | − | − | − | − | − | − | − | − |
| Negative control | + | + | + | + | + | + | + | + | + | + |

As can be seen, a composition of 0.2 wt % maltol, 0.025% benzoin and 0.075% saponin extract showed antimicrobial activity against all tested microorganisms. For E. coli, a clear synergistic effect can be observed for a composition of 0.15 wt % maltol, 0.05 wt % benzoin and 0.15 wt % saponin extract; A. brasiliensis a synergistic effect can be observed for a composition of 0.15 wt % maltol, 0.025 wt % benzoin and 0.08 wt % saponin extract; while for both C. Albicans and S. aureus a clear synergistic effect is demonstrated already for a composition of 0.04 wt % maltol, 0.025 wt % benzoin and 0.075 wt % saponin.

TABLE 5
Agar dilution test results for compositions of saponin (SN) and Styrax
(BZ) extracts with maltol (ML) and citric acid (CIT) (amounts in wt %)

| | Test 1 | | | | | Teat 2 (duplicate) | | | | |
|---|---|---|---|---|---|---|---|---|---|---|
| | AB | CA | PA | EC | SA | AB | CA | PA | EC | SA |
| SN 0.075% + BZ 0.025% + ML 0.05% | + | − | +/− | + | − | + | − | +/− | + | − |
| SN 0.075% + BZ 0.025% + ML 0.05% + CIT 0.015% | + | − | +/− | + | +/− | + | − | +/− | + | +/− |
| SN 0.15% + BZ 0.05% + ML 0.05% | + | − | +/− | + | − | + | − | +/− | + | − |
| SN 0.15% + BZ 0.05% + ML 0.05% + CIT 0.015% | + | − | +/− | + | − | + | − | +/− | + | − |
| SN 0.15% + BZ 0.05% + ML 0.075% | +/− | − | +/− | + | +/− | +/− | − | +/− | + | +/− |
| SN 0.15% + BZ 0.05% + ML 0.075% + CIT 0.015% | +/− | − | +/− | + | − | +/− | − | +/− | + | − |
| SN 0.075% + BZ 0.025% + ML 0.08% | +/− | − | +/− | + | + | +/− | − | +/− | + | + |
| SN 0.075% + BZ 0.025% + ML 0.08% + CIT 0.015% | +/− | − | − | + | +/− | +/− | − | − | + | +/− |
| SN 0.15% + BZ 0.05% + ML 0.08% | +/− | − | +/− | + | − | +/− | − | +/− | + | − |
| SN 0.15% + BZ 0.05% + ML 0.08% + CIT 0.015% | +/− | − | +/− | + | − | +/− | − | +/− | + | − |
| SN 0.075% + BZ 0.025% + ML 0.1% | − | − | − | + | − | − | − | − | + | − |
| SN 0.075% + BZ 0.025% + ML 0.1% + CIT 0.015% | − | − | − | + | − | − | − | − | + | − |
| SN 0.375% + BZ 0.125% + ML 0.2% + CIT 0.015% | − | − | − | − | − | − | − | − | − | − |
| Positive control | − | − | − | − | − | − | − | − | − | − |
| Negative control | + | + | + | + | + | + | + | + | + | + |

As can be seen, a composition of 0.2 wt % maltol, 0.125% benzoin, 0.375% saponin extract and 0.015 wt % citric acid showed antimicrobial activity against all tested microorganisms. For P. aeruginosa, a clear synergistic effect can be observed for a composition of 0.08 wt % maltol, 0.025 wt % benzoin, 0.075 wt % saponin extract, and 0.015 wt % citric acid; while for S. aureus a synergistic effect is demonstrated for a composition of 0.075 wt % maltol, 0.05 wt % benzoin, 0.15 wt % saponin extract and 0.015 wt % citric acid.

### Example 4: Composition of Saponin Extract, Styrax Extract (Benzoin Extract), and Additional Plant Extracts in Agar Dilution Test

An agar dilution test was carried out for various combinations of saponin extract (Sapindus mukorossi) and Styrax extract (Sumatra benzoin), with various additional plant extracts, for evaluating the synergistic antimicrobial effect therebetween. The test protocol was as in Example 3.

The tested extracts were Malpighia emarginata (acerola) fruit extract, Thymus vulgaris (thyme) leaves extract, Origanum vulgare leaves (oregano) extract, Syzygium aromaticum (clove) bud oil, Nigella sativa seeds extract, Pimenta dioica fruit (allspice) extract, Magnolia officinalis bark extract, Cymbopogon citratus (lemongrass) leaves extract, and Paeonia lactiflora extract.

The test results are detailed in Table 6. (+) indicates growth, (+/−) indicates partial growth, while (−) indicates no growth. Positive control was obtained by utilizing a commercial preservative.

TABLE 6
Agar dilution test results for compositions of saponin and
Styrax extracts and various extracts (amounts in wt %)

| | Test results | | | | |
|---|---|---|---|---|---|
| | AB | CA | PA | EC | SA |
| Saponin 0.075% + styrax 0.025% | +/− | +/− | + | + | +/− |
| Allspice 0.2% | − | − | + | + | − |
| Saponin 0.075% + styrax | − | − | +/− | − | − |
| 0.025% + allspice 0.2% | | | | | |
| Magnolia 0.2% | + | + | + | + | + |
| Saponin 0.075% + styrax | − | + | + | + | + |
| 0.025% + magnolia 0.2% | | | | | |
| Lemongrass 0.2% | + | + | + | + | + |
| Saponin 0.075% + styrax | − | − | + | + | − |
| 0.025% + lemongrass 0.2% | | | | | |
| Paeonia 0.2% | + | + | + | + | − |
| Saponin 0.075% + styrax | − | − | + | + | + |
| 0.025% + Paeonia 0.2% | | | | | |
| Positive control | − | − | − | − | − |
| Negative control | + | + | + | + | + |

As can be seen, a composition of 0.075 wt % saponin and styrax 0.025 wt % with Allspice extract 0.2 wt % showed synergism in antimicrobial activity against E. coli and partially for A. brasiliensis. For compositions including 0.2 wt % Magnolia extract, synergism was observed against A. brasiliensis. For compositions including 0.2 wt % Lemongrass extract, synergism was seen against C. albicans, S. aureus and A. brasiliensis. When comprising 0.2 wt % of Paeonia extract, synergism was observed in antimicrobial activity against C. albicans and A. brasiliensis.

### Example 5: Delayed Antimicrobial Activity

A Preservatives Effectiveness Test (PET) was carried out for two compositions: composition 5A that contained 0.2 wt % maltol, 0.05 wt % Styrax extract (Sumatra benzoin) and 0.15 wt % saponin extract (Sapindus mukorossi), and composition 5B that contained 0.2 wt % maltol, 0.125 wt % Styrax extract (Sumatra benzoin), 0.375 wt % saponin extract (Sapindus mukorossi), and 0.015 wt % citric acid. The tests were carried out according to the protocol of Example 1, however inoculated with 5×10⁶ CFU/ml of S. aureus (SA), 5×10⁶ CFU/ml of S. epidermis (SE) and a 6.3×10⁶ CFU/ml mixture of both. These microorganisms are indicative of microorganisms residing naturally on the skin and are part of the desired microbiota of the skin. The log reduction was recorded for various test points, as shown in Table 7.

TABLE 7
Summary of results for compositions 5A and 5B, log reduction
from initial inoculation, in cream formulation

| | 7 hours | | | 24 hours | | | 48 hours | | | 7 days | | |
|---|---|---|---|---|---|---|---|---|---|---|---|---|
| Tested | | | 1:1 | | | 1:1 | | | 1:1 | | | 1:1 |
| composition | SA | SE | mix | SA | SE | mix | SA | SE | mix | SA | SE | mix |
| 5A | 0 | 0 | 0 | 0 | 0 | 0 | 0 | 0 | 0 | 6.69 | 6.70 | 6.80 |
| 5B | 0 | 0 | 0 | 0 | 0 | 0 | 0 | 0 | 0 | 6.69 | 6.70 | 6.80 |
| Cream with | 1.54 | 6.70 | 1.62 | 4.51 | 6.70 | 6.80 | 6.69 | 6.70 | 6.80 | 6.69 | 6.70 | 6.80 |
| synthetic | | | | | | | | | | | | |
| preservative | | | | | | | | | | | | |
| Unpreserved | 0 | 0 | 0 | 0 | 0 | 0 | 0 | 0 | 4.25 | 2.15 | 6.7 | 2.7 |
| cream | | | | | | | | | | | | |

As can be seen, no reduction in microbial population was observed for the first 48 hours in creams that contained compositions 5A and 5B, indicating that only after 48 hours the natural microbiota of the skin is expected to be affected. However, for the synthetically preserved cream, elimination of microbiota is clearly observable starting from several hours after exposure. Thus, the maltol/saponin/Styrax composition (with and without citric acid), unlike the synthetic preservative, is not expected to hinder the natural microbiota for the time period that the formulation normally resides on the skin (several hours), however, as show in Examples 1-3 above, provides antimicrobial activity against the undesired microorganism in the cream.

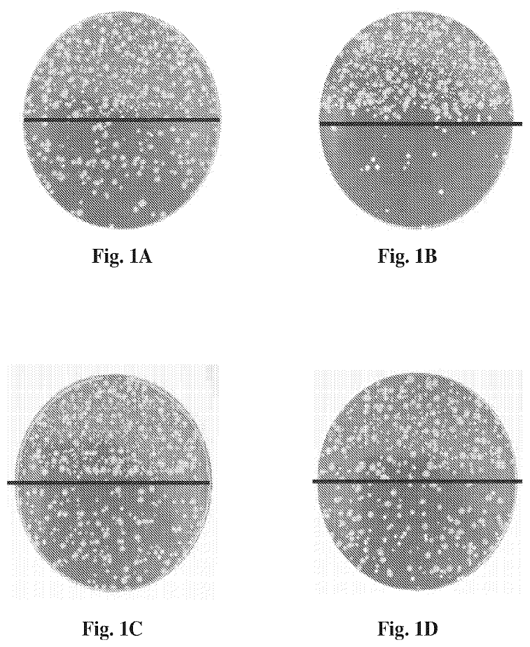

This delayed effect is also demonstrated in FIGS. 1A-1D, which are pictures of an agar plate inoculated with a mixture of S. aureus, S. capitis, S. epidermis and S. hominis, taken after 7 hours of incubation. The mixture of microorganisms represents the natural skin microbiota. FIG. 1A shows a sample of cream without any preservatives. FIG. 1B shows a sample of the cream in which the bottom half of the sample contained a commercial synthetic preservative (ethylhexyl glycerin 1%) and the upper half did not contain a preservative—showing almost complete elimination of the microbiota in the preserved region. In comparison, FIGS. 1C and 1D show samples of the cream with the bottom half of the sample containing the compositions 5A and 5B, respectively, and without preservative in the upper half. Contrary to the commercial preservative, it can be seen that in compositions 5A and 5B, no harm to the natural microbiota is observed, indicating the antimicrobial delayed effect of compositions of the disclosure.

1. An antimicrobial composition comprising:
at least one saponin extract;
an extract from at least one plant species selected from the group consisting of the genus Styrax (a Styrax extract), the genus Myroxylon (a Miroxylon extract), the genus Myrocarpus (a Myrocarpus extract) and mixtures thereof; and
at least one additional plant extract from an additional plant source;
the saponin extract being from a saponin source different from said plant species of the genera Styrax, Myroxylon, or Myrocarpus, and from said additional plant source.

2. (canceled)

3. The composition of claim 1, wherein said at least one additional plant extract is selected from the group consisting of Cinnamomum cassia extract, Paeonia lactiflora extract, Salvia officinalis extract, Magnolia officinalis extract, Commiphora myrrha extract, Boswellia serrata extract, Dipterocarpus turbinatus extract, Copaiba langsdorffii extract, Malpighia glabra or Malpighia emarginata extract, Thymus vulgaris extract, Origanum vulgare extract, Cymbopogon citratus extract, Anethum graveolens extract, Syzygium aromaticum extract, Nigella sativa extract, Pimenta dioica extract, and mixtures thereof.

4. The composition of claim 1, wherein said additional plant extract is maltol extracted from a plant source.

5. The composition of claim 1, wherein the weight ratio between the extract of the additional plant source and the at least one plant extract is between about 1:50 and 50:1.

6. The composition of claim 1, wherein the weight ratio between the saponin extract and the at least one plant extract is between about 1:50 to about 50:1.

7. The composition of claim 1, wherein the weight ratio between the saponin extract and the extract of the additional plant source is between about 1:50 and 50:1.

8. The composition of claim 1, wherein the at least one plant extract contains at least one of cinnamic acid, cinnamic acid derivatives selected from the group consisting of P-coumaryl cinnamate, coniferyl cinnamate, cinnamyl cinnamate, benzyl cinnamate, cinnamic acid esters, and combinations thereof and/or benzoic acid derivatives selected from the group consisting of coniferyl benzoate, cinnamyl benzoate, P-coumaryl benzoate, pinoresinol, benzoic acid esters and combinations thereof, wherein the total amount of cinnamic acid, cinnamic acid derivatives and benzoic acid derivatives in the at least one plant extract is between about 0.001 and 80 wt %.

9.-11. (canceled)

12. The composition of claim 1, wherein the at least one plant extract is Styrax extract obtained from bark or resin of said at least one plant species of the genus Styrax, selected from the group consisting of an extract of Styrax paralleloneurus (Sumatra benzoin), Styrax tonkinensis (Siam Benzoin), Styrax formosanus, Styrax peruvianum, Styrax tolu and mixtures thereof.

13.-16. (canceled)

17. The composition of claim 1, wherein the saponin extract is obtained from a plant source selected from the group consisting of shikakai, soyabeans, beans, peas (Pisum sativum), lucerne, tea, spinach, sugar beet, quinoa, liquorice, sunflower, horse chestnut, ginseng, oats, capsicum peppers, aubergine, tomato seed, alliums, asparagus, yam, fenugreek, yucca and ginseng, lucerne, mung beans, Bupleurum falcatum, Camellia oleifera, Camellia sinensis, Desmodium adscendens, Gypsophila, Panax quinqufolius, Panax japonicas, Quillaja saponaria, Sapindus delavayi, Sapindus mukorossi, Sapindus marginatus, Sapindus saponaria, Sapindus trifoliatus, Saponaria officinalis, Yucca schidigera, and any mixture thereof.

18. The composition of claim 17, wherein the saponin extract is obtained from a plant source selected from the group consisting of Camellia oleifera, Camellia sinensis, Quillaja saponaria, Sapindus mukorossi, Sapindus saponaria, Saponaria officinalis, and any mixture thereof.

19. (canceled)

20. The composition of claim 1, wherein the composition comprises saponin extract in a content of between about 0.001 wt % and 2 wt %.

21. The composition of claim 1, comprising at antimicrobial effective amount of a mixture of agents, the mixture of agents consisting of said at least one saponin extract, said at least one plant extract from at least one plant species selected from the genus Styrax, Myroxylon and/or Myrocarpus and mixtures thereof, and said at least one additional plant extract from an additional plant source.

22. The antimicrobial composition of claim 1 comprising
a saponin extract selected from Camellia oleifera extract, Camellia sinensis extract, Quillaja saponaria extract, Sapindus mukorossi extract, Sapindus saponaria extract, and Saponaria officinalis extract, and any mixture thereof,
a Styrax extract from Styrax paralleloneurus (Sumatra benzoin), Styrax tonkinensis (Siam Benzoin), Styrax formosanus, Styrax peruvianum, Styrax tolu and mixtures thereof, and
at least one plant extract from an additional plant source selected from Cinnamomum extract, Paeonia extract, Salvia extract, Magnolia extract, Commiphora extract, Boswellia extract, Dipterocarpus extract, Copaiba extract, Malpighia or Thymus extract, Origanum extract, Cymbopogon extract, Anethum extract, Syzygium extract, Nigella extract, Pimenta extract, and mixtures thereof;
and optionally at least one additive.

23. An add-on antimicrobial composition consisting of a combination of:
at least one saponin extract;
at least one plant extract from at least one plant species selected from the group consisting of the genus Styrax, Myroxylon and/or Myrocarpus and mixtures thereof; and
at least one additional plant extract from an additional plant source;
the saponin extract being from a saponin source different from said at least one plant species and from said additional plant source.

24. (canceled)

25. The composition of claim 1, formulated as a preservative formulation, an antimicrobial formulation, a pharmaceutical composition, a disinfectant formulation and a cosmetic formulation.

26. The composition according to claim 1, said composition selected from the group consisting of:
(a) a composition comprising saponin extract, Styrax extract and maltol;
(b) a composition comprising saponin extract, Styrax extract, maltol, and citric acid;
(c) a composition comprising saponin extract, Styrax extract and Cinnamomum cassia extract;
(d) a composition comprising saponin extract, Styrax extract, Cinnamomum cassia extract and maltol;
(e) a composition comprising saponin extract, Styrax extract, and Magnolia Officinalis extract;
(f) a composition comprising saponin extract, Styrax extract, and Paeonia Lactiflora extract;
(g) a composition comprising saponin extract, Styrax extract, Cinnamomum cassia extract, citric acid and maltol;
(h) a composition comprising saponin extract, Styrax extract, Cinnamomum cassia extract, and citric acid;
(i) a composition comprising saponin extract, Styrax extract, Cinnamomum cassia extract, Paeonia Lactiflora extract, citric acid and maltol;
(j) a composition comprising saponin extract, Styrax extract, Cinnamomum cassia extract, and Salvia officinalis extract;
(k) a composition comprising saponin extract, Styrax extract, Cinnamomum cassia extract, and shikimic acid;
(l) a composition comprising saponin extract, Styrax extract, and Nigella sativa extract;
(m) a composition comprising saponin extract, Styrax extract, and Pimenta dioica extract;
(n) a composition comprising saponin extract, Styrax extract, and Cymbopogon citrus extract;
(o) a composition comprising saponin extract, Styrax extract, and Salvia officinalis leaves extract;
(p) a composition comprising saponin extract, Styrax extract, and Malpighia emarginata extract
(q) a composition comprising saponin extract, Styrax extract, and Thymus vulgaris extract;
(r) a composition comprising saponin extract, Styrax extract, and Origanum vulgare extract; and
(s) a composition comprising saponin extract, Styrax extract, and Syzygium aromaticum extract.

27. The composition of claim 1, having a delayed antimicrobial activity, wherein the composition shows antimicrobial activity observable from at least 5 hours of continuous exposure of microorganisms to the composition.

28.-32. (canceled)

33. The cosmetic product comprising the composition as defined in claim 1.

34. The cosmetic product of claim 33, wherein the cosmetic product is selected from the group consisting of soap, a hair care product, an oral care product and a skin care product.
